SEQUENCE LISTING (15 sequences)

SEQ ID NO: 1 - protein, 128 aa, Homo sapiens
KPKGMTSSQWFKIQHMQPSPQACNSAMKNINKHTKRCKDLNTFLHEPFSSVAATCQTPKIACKNGDKNCHQSHGPVSLTMCKLTSGKYPNCRYKEKRQNKSYVVACKPPQKKDSQQFHLVPVHLDRVL

SEQ ID NO: 2 - protein, 45 aa, Homo sapiens
KPKGMTSSQWFKIQHMQPSPQACNSAMKNINKHTKRCKDLNTFLH

SEQ ID NO: 3 - protein, 71 aa, Homo sapiens
KPKGMTSSQWFKIQHMQPSPQACNSAMKNINKHTKRCKDLNTFLHEPFSSVAATCQTPKIACKNGDKNCHQ

SEQ ID NO: 4 - protein, 83 aa, Homo sapiens
KPKGMTSSQWFKIQHMQPSPQACNSAMKNINKHTKRCKDLNTFLHEPFSSVAATCQTPKIACKNGDKNCHQSHGPVSLTMCKL

SEQ ID NO: 5 - protein, 97 aa, Homo sapiens
KPKGMTSSQWFKIQHMQPSPQACNSAMKNINKHTKRCKDLNTFLHEPFSSVAATCQTPKIACKNGDKNCHQSHGPVSLTMCKLTSGKYPNCRYKEKR

SEQ ID NO: 6 - protein, 107 aa, Homo sapiens
KPKGMTSSQWFKIQHMQPSPQACNSAMKNINKHTKRCKDLNTFLHEPFSSVAATCQTPKIACKNGDKNCHQSHGPVSLTMCKLTSGKYPNCRYKEKRQNKSYVVACK

SEQ ID NO: 7 - protein, 52 aa, Homo sapiens
EPFSSVAATCQTPKIACKNGDKNCHQSHGPVSLTMCKLTSGKYPNCRYKEKR

SEQ ID NO: 8 - protein, 22 aa, Artificial Sequence
  histidine peptide tag for purification purposes
MSYYHHHHHHLESTSLYKKAGF

SEQ ID NO: 9 - protein, 150 aa, Artificial Sequence
  analog of RNASE 7 peptide fragment
MSYYHHHHHHLESTSLYKKAGFKPKGMTSSQWFKIQHMQPSPQACNSAMKNINKHTKRCKDLNTFLHEPFSSVAATCQTPKIACKNGDKNCHQSHGPVSLTMCKLTSGKYPNCRYKEKRQNKSYVVACKPPQKKDSQQFHLVPVHLDRVL

SEQ ID NO: 10 - protein, 67 aa, Artificial Sequence
  analog of RNASE 7 peptide fragment
MSYYHHHHHHLESTSLYKKAGFKPKGMTSSQWFKIQHMQPSPQACNSAMKNINKHTKRCKDLNTFLH

SEQ ID NO: 11 - protein, 93 aa, Artificial Sequence
  analog of RNASE 7 peptide fragment
MSYYHHHHHHLESTSLYKKAGFKPKGMTSSQWFKIQHMQPSPQACNSAMKNINKHTKRCKDLNTFLHEPFSSVAATCQTPKIACKNGDKNCHQ

SEQ ID NO: 12 - protein, 105 aa, Artificial Sequence
  analog of RNASE 7 peptide fragment
MSYYHHHHHHLESTSLYKKAGFKPKGMTSSQWFKIQHMQPSPQACNSAMKNINKHTKRCKDLNTFLHEPFSSVAATCQTPKIACKNGDKNCHQSHGPVSLTMCKL

SEQ ID NO: 13 - protein, 118 aa, Artificial Sequence
  analog of RNASE 7 peptide fragment
MSYYHHHHHHLESTSLYKKAGFKPKGMTSSQWFKIQHMQPSPQACNSAMKNINKHTKRCKDLNTFLHEPFSSVAATCQTPKIACKNGDKNCHQSHGPVSLTMCKLTSGKYPNCRYKEK

SEQ ID NO: 14 - protein, 129 aa, Artificial Sequence
  analog of RNASE 7 peptide fragment
MSYYHHHHHHLESTSLYKKAGFKPKGMTSSQWFKIQHMQPSPQACNSAMKNINKHTKRCKDLNTFLHEPFSSVAATCQTPKIACKNGDKNCHQSHGPVSLTMCKLTSGKYPNCRYKEKRQNKSYVVACK

SEQ ID NO: 15 - protein, 74 aa, Artificial Sequence
  analog of RNASE 7 peptide fragment
MSYYHHHHHHLESTSLYKKAGFEPFSSVAATCQTPKIACKNGDKNCHQSHGPVSLTMCKLTSGKYPNCRYKEKR

(19) United States
(12) Patent Application Publication
(10) Pub. No.: US 2015/0072922 A1
(43) Pub. Date: Mar. 12, 2015

(54) RNASE 7 ANTIMICROBIAL PEPTIDES

(71) Applicant: THE RESEARCH INSTITUTE AT NATIONWIDE CHILDREN'S HOSPITAL, Columbus, OH (US)

(72) Inventors: David Hains, Memphis, TN (US); Andrew Schwaderer, Blacklick, OH (US); Huanyu Wang, Dublin, OH (US)

(21) Appl. No.: 14/391,308

(22) PCT Filed: Apr. 17, 2013
(86) PCT No.: PCT/US13/36993

§ 371 Date: Oct. 8, 2014

Related U.S. Application Data
(60) Provisional application No. 61/625,153, filed on Apr. 17, 2012.
(60) Provisional application No. 61/709,807, filed on Oct. 4, 2012.

(51) Int. Cl. A61K 38/17; A61L 29/16; A61L 31/16; A61L 31/04; C07K 14/47; A61K 45/06

(52) U.S. Cl. CPC A61K 38/1709; C07K 14/47; A61K 45/06; A61L 31/16; A61L 31/047; A61L 29/16; A61L 2300/404
USPC 514/2.6; 530/350; 514/2.3; 514/2.4; 514/2.8; 514/2.7

(57) ABSTRACT

Described herein are compounds and compositions comprising antimicrobial peptides and methods for their use.

### CROSS-REFERENCE TO RELATED APPLICATIONS

This application claims priority under 35 U.S.C. §119 (e) to U.S. Provisional Application Ser. No. 61/625,153 filed Apr. 17, 2012 and U.S. Provisional Application Ser. No. 61/709,807 filed Oct. 4, 2012, both of which are expressly incorporated herein by reference.

### INCORPORATION BY REFERENCE OF MATERIAL SUBMITTED ELECTRONICALLY

Incorporated by reference in its entirety is a computer-readable sequence listing submitted concurrently herewith and identified as follows: one 13,296 byte ASCII (text) file named “1393172_{—}1. TXT,” created on Apr. 17, 2013.

### TECHNICAL FIELD

This invention relates to the use of peptide fragments based on the peptide sequences of Ribonuclease 7 (RNase 7) with antimicrobial activity.

### BACKGROUND

Currently, urinary tract infections (UTIs) account for a large health care burden in all age groups. Annually in the United States, UTIs are responsible for 9 million physician visits, 100,000 hospitalizations, direct and indirect costs of over 3.5 billion dollars. Long-term complications of UTIs can include renal scarring, hypertension, chronic kidney disease and complications in future pregnancies. Further, UTIs with multi-drug resistant microbes have increased dramatically in recent years. Antibiotics are increasingly insufficient for treatment of UTIs highlighting the importance of novel management strategies.

Antimicrobial peptides (AMPs): AMPs are small proteins that possess antimicrobial activity against bacteria, enveloped viruses, fungi, and some protozoa. AMPs are usually cationic secondary to the presence of lysine and/or arginine residues and amphipathic, which allows them to concentrate in both a membrane and aqueous environment. AMPs may be constitutively expressed and/or induced by invading pathogens. Although many AMPs have been described in other organ systems, few have been studied in the human urinary tract. Because pathogenic bacteria are still susceptible to endogenous AMPs, they have been considered a possible treatment against drug resistant organisms. Ultimately, most AMPs destabilize bacterial lipid bilayers. Unfortunately, our understanding of the fundamental mechanisms of AMP action and structure-functional relationship with bacteria is cursory.

Very little is known how the human urinary tract maintains sterility. One way sterility is maintained is via the innate immune system's antimicrobial peptides (AMPs). It has been previously described that an AMP, known as Ribonuclease 7 (RNase 7), is produced throughout the urinary tract and that concentrations of RNase 7 present in urine are sufficient for antimicrobial activity. Also, bacterial growth is enhanced in urine when endogenous RNase 7 is neutralized. Little is known of the properties responsible for the target specificity and selective toxicity of RNase 7 despite its potency.

The antimicrobial peptide Ribonuclease 7 (RNase7) is expressed in kidney and bladder epithelia. RNase7 plays a critical role in maintaining urinary tract sterility and possesses antimicrobial properties against gram-negative and gram-positive uropathogenic bacteria. The mechanisms of the antimicrobial activity of RNase7 involves binding and depolarizing the bacterial membrane, a function that is different from those of the conventional antibiotics. Therefore, RNase7 and various peptide fragment thereof are disclosed herein for use as a therapeutic agents for treating bacterial infection either as the single active agent, or in combination with other anti-bacterial or anti-microbial agents.

### SUMMARY

In one aspect, the present invention provides a method for treating a subjecting having a bacterial infection by administering a therapeutically effective amount of at least one RNase 7 peptide fragment to the subject. The subject can be a human subject, and the one or more RNase 7 peptide fragments can be administered in a pharmaceutically acceptable carrier. The bacterial infection can be by a gram-positive bacteria, or by a gram-negative bacteria. The RNase 7 peptide fragment can be an N-terminal fragment, a C-terminal fragment, or a middle peptide fragment lacking amino acids from each end. For example, the RNase 7 peptide fragment can be an N-terminal fragment lacking up to 30 amino acids from the C-terminal end.

Another aspect of the invention provides RNase 7 peptide fragments having antimicrobial activity equivalent or enhanced activity relative to the native RNase 7 protein (SEQ ID NO: 1). RNase 7 peptide fragments are defined herein as amino acid sequences that are lacking at least one amino acid relative to the full RNase 7 protein of SEQ ID NO: 1. The peptide fragment can be an N-terminal fragment, a C-terminal fragment, or a middle peptide fragment lacking amino acids from each end. For example, the RNase 7 peptide fragment can be an N-terminal fragment lacking up to 30 amino acids from the C-terminal end. The RNase 7 peptide fragment can include three α-helixes. In various embodiments, the RNase 7 peptide fragment can include at least 45, at least 50, at least 55, at least 60, at least 65, at least 70, at least 75, at least 80, at least 85, at least 90, at least 95, or at least 100 amino acids. In one embodiment the peptide fragment comprises a 10, 20, 30, 45, 50, 60, 70, 80, 90 or 100 contiguous amino acid sequence identical to a sequence present in the first 107 amino acids of SEQ ID NO: 1 but excluding a 10 amino acid sequence or longer sequence that is identical to a contiguous sequence present in amino acids 108-128 of SEQ ID NO: 1. In one embodiment a composition is provided comprising two or more fragments of RNase 7 wherein the fragments differ in amino acid sequence and do not comprise a 10 amino acid sequence or longer sequence that is identical to a contiguous sequence present in amino acids 108-128 of SEQ ID NO: 1. In one embodiment the two or more different fragments of RNase 7 are linked to one another. In further embodiments, the RNase 7 peptide fragments are modified relative to the corresponding sequence in SEQ ID NO: 1 to include one or more conservative amino acid substitutions.

### BRIEF DESCRIPTION OF THE DRAWINGS

FIG. 1 Bacterial cell death assay results for C-terminal (top panel), N-terminal (middle panel), and middle (bottom panel) fragments for E. coli. (Top) C-terminal fragments result in reduced membrane permeabilization and death for E. coli compared to full-length protein. (Middle) The N-terminal fragment F:1-97 has improved activity compared to the full-length protein. (Bottom) The middle fragment F:46-97 demonstrates enhanced permeabilization and death compared to the full-length protein.

FIG. 2 Bacterial cell death assay results for C-terminal (top panel), N-terminal (middle panel) and middle (bottom panel) fragments for S. saprophyticus. Top Panel: C-terminal fragments result in reduced membrane permeabilization and death for S. saprophyticus compared to full-length protein. Middle Panel: The N-terminal fragment F:1-97 has improved activity compared to full-length. Bottom Panel: Unlike its enhanced activity against E. coli, the middle fragment F:46-97 demonstrates no activity compared to full length protein.

FIG. 3 E. coli growth curves after treatment with different recombinant peptides at various pH levels. (A) E. coli growth is optimum at pH 5 (gray squares) compared to pH 7 and 9. (B) pH 9 (dotted line) decreases full-length RNase 7 activity, as evidenced by enhanced growth compared to that seen at pH 7. (C) F:1-97 has an activity pattern comparable to full-length peptide at various pHs. (D) F:72-128 demonstrates similar activity against E. coli at all pHs.

FIG. 4 E. coli growth curves after treatment with different recombinant peptides at various NaCl titers and with no added NaCl (gray squares). (A) E. coli demonstrated improved growth with 50 and 150 mM NaCl. Growth was completely inhibited with NaCl at 500 mM. (B) Full-length RNase 7 activity was not different at 50 and 150 mM NaCl. Bacterial growth was enhanced at low concentrations of NaCl, much like the conditions with no additional peptide (top left panel). (C) F:1-97 has activity pattern comparable to full-length peptide at various NaCl concentrations. (D) F:72-128 has similar activity against E. coli at all NaCl concentrations except 500 mM. Interestingly, growth is enhanced at 500 mM NaCl with F:72-128 compared to all other conditions.

FIG. 5 E. coli growth curves after treatment with different recombinant peptides at various CaCl₂ titers and with no added CaCl₂ (gray squares). (A) E. coli showed similar growth under all conditions. (B) Full-length RNase 7 activity is inversely related to CaCl₂ concentration. (C) F:1-97 has activity pattern comparable to full-length peptide at various CaCl₂ concentrations. (D) F:72-128 has similar activity against E. coli at all CaCl₂.

FIG. 6 S. saprophyticus growth curves after treatment with different recombinant peptides at various pH levels. A: S. saprophyticus growth is optimum at pH 9 (dashed line) compared to pH 5 and 7. B: Full-length RNase 7 activity is potent at all pH's. C: F:1-97 has optimal activity at pH 5 (gray squares). D: F:72-128 has minimal activity with enhanced growth at pH 7 and 9.

FIG. 7 S. saprophyticus growth curves after treatment with different recombinant peptides at various NaCl titers and with no added NaCl (gray squares). Panel A: S. saprophyticus has improved growth with 50 mM NaCl and 150 mM NaCl. Growth is blunted compared to the other NaCl concentrations at NaCl 500 mM (black boxes) but still better than no added salt (gray boxes). Panel B: Full length RNase 7 activity inversely related to NaCl concentration. At 500 mM, activity is markedly diminished. Panel C: F:1-97 has activity pattern comparable to full-length peptide at various NaCl concentrations. Panel D: F:72-128 has minimal activity against S. saprophyticus at all NaCl concentrations and does not differ from bacterial growth with no recombinant peptide added (Panel A).

FIG. 8 S. saprophyticus growth curves after treatment with different recombinant peptides at various CaCl₂ titers and with no added CaCl₂ (gray squares). Panel A: S. saprophyticus has markedly increased growth with all concentrations of additional CaCl₂. Panel B: Full length RNase 7 activity is inversely related to CaCl₂ concentration. Activity significantly diminished at highest CaCl₂ concentration. Panel C: F:1-97 activity is inversely related to CaCl₂ concentration but more pronounced compared to full length RNase 7. Panel D: F:72-128 has similar minimal activity against E. coli at all CaCl₂.

FIG. 9 Proteus mirabilis growth curves after treatment with different recombinant peptides at various pH levels. Panel A: P. mirabilis growth is least at pH 5 (gray squares) and increases as pH increases. Panel B: Full length RNase 7 activity is potent at pH 5. At pH 7 and 9, full length activity is significantly reduced. Panel C: F:1-97 has optimal activity at pH 5 (gray squares), but less compared to full length (Panel B). Panel D: F:72-128 has minimal activity with enhanced growth at pH 7 and 9.

FIG. 10 Proteus mirabilis growth curves after treatment with different recombinant peptides at various NaCl titers and with no added NaCl (gray squares). Panel A: P. mirabilis has improved growth at all NaCl concentrations compared to no added salt (gray boxes). Panel B: Full length RNase 7 activity markedly diminished at all salt concentrations. Panel C: F:1-97 has activity pattern comparable to full-length peptide at various NaCl concentrations. Interestingly, F:1-97 has less activity than full length RNase 7 (Panel B). Activity overall is similar to control conditions with no recombinant peptide added (top left). Panel D: F:72-128 has minimal activity against P. mirabilis at all NaCl concentrations and does not differ from bacterial growth with no recombinant peptide added (Panel A).

FIG. 11 Proteus mirabilis growth curves after treatment with different recombinant peptides at various CaCl₂ titers and with no added CaCl₂ (gray squares). Panel A: CaCl₂ concentration has little effect on P. mirabilis growth except at highest concentration. At 16.25 mM (black squares), P. mirabilis has slightly enhanced growth compared to other conditions. Panel B: Full length RNase 7 activity is significantly diminished at higher CaCl₂ concentrations but relatively unaffected at 1.25 mM concentration (black line). Panel C: F:1-97 activity is not significantly altered by CaCl₂ concentrations. Further, F:1-97 has minimal activity against P. mirabilis. Panel D: F:72-128 has similar minimal activity against P. mirabilis at all CaCl₂.

FIG. 12 Representation of structural elements with N-terminal, C-terminal, and middle fragments. Fragment nomenclature example: RNase 7 fragment amino acids 1 to 45=F:1-45.

FIG. 13 Atomic Force Microscopy (AFM) images showing morphological effect of RNase 7 on E. Coli, P. aeruginosa and E. faecalis. The high-resolution images demonstrate nanometer-scale changes in cell morphology including pores, spore rodlets and bacterial division septa. Ultra-pure water was used as a negative control. AFM imaging was performed with the Multimode AFM instrument (Veeco, Santa Barbara, Calif.).

The present invention provides small peptides based on the sequences of RNase7 with antimicrobial activity. Preferably, the antimicrobial RNase 7 peptide fragments have higher antimicrobial activity and lower cytotoxicity activity relative to full length RNase 7.

The terminology as set forth herein is for description of the embodiments only and should not be construed as limiting of the invention as a whole. As used in the description of the invention and the appended claims, the singular forms “a”, “an”, and “the” are inclusive of their plural forms, unless contraindicated by the context surrounding such.

A disease or disorder is “alleviated” if the severity of a symptom of the disease, condition, or disorder, or the frequency with which such a symptom is experienced by a subject, or both, are reduced.

As used herein, the term “subject” refers to an individual (e.g., human, animal, or other organism) to be treated by the methods or compositions of the present invention. Subjects include, but are not limited to, mammals (e.g., murines, simians, equines, bovines, porcines, canines, felines, and the like), and includes humans. In the context of the invention, the term “subject” generally refers to an individual who will receive or who has received treatment for a condition characterized by the presence of bacteria (e.g., Bacillus anthracis (e.g., in any stage of its growth cycle), or in anticipation of possible exposure to bacteria. As used herein, the terms “subject” and “patient” are used interchangeably, unless otherwise noted.

As used herein, the term “treating a surface” refers to the act of exposing a surface to one or more compositions of the present invention. Methods of treating a surface include, but are not limited to, spraying, misting, submerging, wiping, and coating. Surfaces include organic surfaces (e.g., food products, surfaces of animals, skin, etc.) and inorganic surfaces (e.g., medical devices, countertops, instruments, articles of commerce, clothing, etc.).

As used herein, the term “therapeutically effective amount” refers to the amount that provides a therapeutic effect, e.g., an amount of a composition that is effective to treat or prevent pathological conditions, including signs and/or symptoms of disease, associated with a pathogenic organism infection (e.g., germination, growth, toxin production, etc.) in a subject.

The terms “bacteria” and “bacterium” refer to all prokaryotic organisms, including those within all of the phyla in the Kingdom Procaryotae. As used herein, the term “microorganism” refers to any species or type of microorganism, including but not limited to, bacteria, archaea, fungi, protozoans, mycoplasma, and parasitic organisms.

As used herein the term “colonization” refers to the presence of bacteria in a subject that are either not found in healthy subjects, or the presence of an abnormal quantity and/or location of bacteria in a subject relative to a healthy patient.

“Amino acid” is used herein to refer to a chemical compound with the general formula: H₂N—CHR—COOH, where R, the side chain, is H or an organic group. Where R is organic, R can vary and is either polar or nonpolar (i.e., hydrophobic). The amino acids of this invention can be naturally occurring or synthetic (often referred to as nonproteinogenic). As used herein, an organic group is a hydrocarbon group that is classified as an aliphatic group, a cyclic group or combination of aliphatic and cyclic groups. The term “aliphatic group” means a saturated or unsaturated linear or branched hydrocarbon group. This term is used to encompass alkyl, alkenyl, and alkynyl groups, for example. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” refers to mono- or polycyclic aromatic hydrocarbon groups. As used herein, an organic group can be substituted or unsubstituted. One letter and three letter symbols are used herein to designate the naturally occurring amino acids. Such designations including R or Arg, for Arginine, K or Lys, for Lysine, G or Gly, for Glycine, and X for an undetermined amino acid, and the like, are well known to those skilled in the art.

The terms “polypeptide” and “peptide” as used herein, are used interchangeably and refer to a polymer of amino acids. These terms do not connote a specific length of a polymer of amino acids. Thus, for example, the terms oligopeptide, protein, and enzyme are included within the definition of polypeptide or peptide, whether produced using recombinant techniques, chemical or enzymatic synthesis, or naturally occurring. This term also includes polypeptides that have been modified or derivatized, such as by glycosylation, acetylation, phosphorylation, and the like.

As used herein, the term “conservative amino acid substitution” generally refers to exchanges within one of the following five groups:
I. Small aliphatic, nonpolar or slightly polar residues: Ala, Ser, Thr, Pro, Gly;
II. Polar, charged residues and their amides: Asp, Asn, Glu, Gln, His, Arg, Lys;
III. Large, aliphatic, nonpolar residues: Met Leu, Ile, Val, Cys;
IV. Large, aromatic residues: Phe, Tyr, Trp.

The term “hydrophobic amino acid side chain” or “nonpolar amino acid side chain,” is used herein to refer to amino acid side chains having properties similar to oil or wax in that they repel water. In water, these amino acid side chains interact with one another to generate a nonaqueous environment. Examples of amino acids with hydrophobic side chains include, but are not limited to, valine, leucine, isoleucine, phenylalanine, and tyrosine.

The term “polar amino acid side chain” is used herein to refer to groups that attract water or are readily soluble in water or form hydrogen bonds in water. Examples of polar amino acid side chains include hydroxyl, amine, guanidinium, amide, and carboxylate groups. Polar amino acid side chains can be charged or non-charged.

The term “non-charged polar amino acid side chain” or “neutral polar amino acid side chain” is used herein to refer to amino acid side chains that are not ionizable or do not carry an overall positive or negative charge. Examples of amino acids with non-charged polar or neutral polar side chains include serine, threonine, glutamine, and the like.

The term “positively charged amino acid side chain” refers to amino acid side chains that are able to carry a full or positive charge and the term “negatively charged amino acid side chain” refers to amino acid side chains that are able to carry a negative charge. Examples of amino acids with positively charged side chains include arginine, histidine, lysine, and the like. Examples of amino acids with negatively charged side chains include aspartic acid and glutamic acid, and the like.

A “fragment” or “segment” of a referenced amino acid sequence is intended to designate an amino acid sequence of length shorter than the referenced sequence, comprising at least one amino acid, or a portion of a nucleic acid sequence comprising at least one nucleotide. The terms “fragment” and “segment” are used interchangeably herein.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

The term “inhibit,” as used herein, refers to the ability of a compound or any agent to reduce or impede a described function or pathway. For example, inhibition can be by at least 10%, by at least 25%, by at least 50%, and even by at least 75%.

An “isolated” compound/moiety is a compound/moeity that has been removed from components naturally associated with the compound/moiety. For example an isolated peptide is free of nucleic acids and other cellular components.

As used herein, the term “purified” and like terms refer to, and define, an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein the term “peptidomimetic” refers to a chemical compound having a structure that is different from the general structure of an existing peptide, but that functions in a manner similar to the existing peptide, e.g., by mimicking the biological activity of that peptide. Peptidomimetics typically comprise naturally-occurring amino acids and/or unnatural amino acids, but can also comprise modifications to the peptide backbone. For example a peptidomimetic may include one or more of the following modifications:

1. peptides wherein one or more of the peptidyl —C(O)NR— linkages (bonds) have been replaced by a non-peptidyl linkage such as a —CH2-carbamate linkage (—CH2OC(O)NR—), a phosphonate linkage, a —CH2-sulfonamide (—CH2-S(O)2NR—) linkage, a urea (—NHC(O)NH—) linkage, a —CH2-secondary amine linkage, an azapeptide bond (CO substituted by NH), or an ester bond (e.g., depsipeptides, wherein one or more of the amide (—CONHR—) bonds are replaced by ester (COOR) bonds) or with an alkylated peptidyl linkage (—C(O)NR—) wherein R is C1-C4 alkyl;

2. peptides wherein the N-terminus is derivatized to a —NRR1 group, to a —NRC(O)R group, to a —NRC(O)OR group, to a —NRS(O)2R group, to a —NHC(O)NHR group where R and R1 are hydrogen or C1-C4 alkyl with the proviso that R and R1 are not both hydrogen;

3. peptides wherein the C terminus is derivatized to —C(O)R2 where R2 is selected from the group consisting of C1-C4 alkoxy, and —NR3R4 where R3 and R4 are independently selected from the group consisting of hydrogen and C1-C4 alkyl;

4. modification of a sequence of naturally-occurring amino acids with the insertion or substitution of a non-peptide moiety, e.g. a retroinverso fragment.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

A urinary tract infection can include an infection of the bladder, kidneys, and/or ureter, and the like, including cystitis, pyleonephritis, and urethritis. Illustrative urinary tract infections can include infections by one or more organisms, for example, organisms selected from Escherichia, Staphylococcus, Proteus Klebsiella, Enterococcus, Proteus, Morganella, Pseudomonas, Group B Streptococcus, Candida, BK virus, Cytomegalovirus (CMV), Epstein-Barr virus (EBV), and the like.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

As used herein an “amino acid modification” refers to a substitution, addition or deletion of an amino acid, and includes substitution with, or addition of, any of the 20 amino acids commonly found in human proteins, as well as unusual or non-naturally occurring amino acids. Commercial sources of unusual amino acids include Sigma-Aldrich (Milwaukee, Wis.), ChemPep Inc. (Miami, Fla.), and Genzyme Pharmaceuticals (Cambridge, Mass.). Unusual amino acids may be purchased from commercial suppliers, synthesized de novo, or chemically modified or derivatized from naturally occurring amino acids. Amino acid modifications include linkage of an amino acid to a conjugate moiety, such as a hydrophilic polymer, acylation, alkylation, and/or other chemical derivatization of an amino acid.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

The amino acid sequence for full length Rnase 7 is KPKGM TSSQW FKIQH MQPSP QACNS AMKNI NKHTK RCKDL NTFLH EPFSS VAATC QTPKI ACKNG DKNCH QSHGP VSLTM CKLTS GKYPN CRYKE KRQNK SYVVA CKPPQ KKDSQ QFHLV PVHLD RVL (SEQ ID NO: 1).

The antimicrobial peptides disclosed herein can be further modified in a variety of ways to form derivatives. These modifications include addition of organic groups to form modified polypeptides, or addition, substitution or deletion of amino acids. These modifications preferably do not eliminate or substantially reduce the biological activity of the peptide. The biological activity of a polypeptide can be determined, for example, as described in the Examples section. Conservative amino acid substitutions typically can be made without affecting biological activity.

In one embodiment antimicrobial derivative peptides of SEQ ID NO: 1 are provided wherein the derivative peptide comprises a peptide that differs from SEQ ID NO: 1 by 1, 2, 3, 4, 5, 6, 7, 8 or 9 amino acid modifications. In one embodiment the amino acid modifications are located in the C-terminal region of SEQ ID NO: 1 extending from amino acid 108 to the C-terminus. In one embodiment the amino acid modifications are amino acid substitutions, and in a further embodiment the derivative peptides comprise a peptide sequence that differs from SEQ ID NO: 1 by 1, 2, 3, 4, 5, 6, 7, 8 or 9 conservative amino acid substitutions in the C-terminal region of SEQ ID NO: 1 extending from amino acid 108 to 128. In accordance with one embodiment the derivative peptide comprises a contiguous fragment of SEQ ID NO: 1 consisting of amino acid positions 1-45 (SEQ ID NO: 2), 1-71 (SEQ ID NO: 3), 1-83 (SEQ ID NO: 4), 1-97 (SEQ ID NO: 5), 1-107 (SEQ ID NO: 6) or 43-97 (SEQ ID NO: 7), with the proviso that derivative peptide does not consist of SEQ ID NO: 1. In a further embodiment the derivative peptide does not comprise a 10 amino acid sequence that is identical to a contiguous 10 amino acid sequence present in amino acids 108-128 of SEQ ID NO: 1.

In accordance with one embodiment an antimicrobial composition is provided, wherein the composition comprises an isolated peptide, said peptide comprising a sequence selected from SEQ ID NO: 5, SEQ ID NO: 6 or SEQ ID NO: 7, or ii) a peptide having at least 90% amino acid sequence identity with SEQ ID NO: 5, SEQ ID NO: 6, or SEQ ID NO: 7, or a peptidomimetic derivative of i) or ii), with the proviso that said peptide does not consist of SEQ ID NO: 1 and/or comprise a 10 amino acid sequence that is identical to a contiguous 10 amino acid sequence present in amino acids 108-128 of SEQ ID NO: 1. In one embodiment the peptide comprises a sequence selected from i) SEQ ID NO: 5, SEQ ID NO: 6 or SEQ ID NO: 7 or a peptide having at least 95% amino acid sequence identity with SEQ ID NO: 5, SEQ ID NO: 6 or SEQ ID NO: 7. In one embodiment a composition is provided comprising an isolated peptide of SEQ ID NO: 5, with the proviso that the isolated peptide does not comprise a 10 amino acid sequence that is identical to a contiguous 10 amino acid sequence present in amino acids 108-128 of SEQ ID NO: 1. In one embodiment a composition is provided comprising an isolated peptide of SEQ ID NO: 7, with the proviso that the isolated peptide does not comprise a 10 amino acid sequence that is identical to a contiguous 10 amino acid sequence present in amino acids 108-128 of SEQ ID NO: 1. In one embodiment the composition comprises two or more peptide fragments of SEQ ID NO; 1, including for example two or more peptide fragments selected from the group consisting of SEQ ID NO: 2, SEQ ID NO: 4, SEQ ID NO: 5, SEQ ID NO: 6 or SEQ ID NO: 7. In one embodiment the peptides are linked to one another. In a further embodiment the peptides are modified by linking a heterologus moiety to the peptide. In one embodiment the peptides are linked to polyethylene glycol. In accordance with one embodiment the antimicrobial composition further comprises a supplemental anti-microbial agent, including for example an antibiotic.

In some embodiments, the peptide of the present disclosures comprises a non-native amino acid sequence which has at least 75%, 80%, 85%, 90% or 95% sequence identity to an amino acid sequence of SEQ ID NO: 2, SEQ ID NO: 4, SEQ ID NO: 5, SEQ ID NO: 6 or SEQ ID NO: 7, or a peptidomimetic derivative of SEQ ID NO: 2, SEQ ID NO: 4, SEQ ID NO: 5, SEQ ID NO: 6 or SEQ ID NO: 7. The statement that the peptide is a non-native is intended to exclude the native peptide of SEQ ID NO: 1. In some embodiments, the peptide of the present disclosures comprises a non-native amino acid sequence which has at least 75%, 80%, 85%, 90% or 95% sequence identity to an amino acid sequence of SEQ ID NO: 5, SEQ ID NO: 6 or SEQ ID NO: 7, or peptidomimetic derivative of SEQ ID NO: 5, SEQ ID NO: 6 or SEQ ID NO: 7. In some embodiments, the peptide of the present disclosure comprises an amino acid sequence which has at least a 90% amino acid sequence identity with SEQ ID NO: 5 or SEQ ID NO: 7, with the proviso that the peptide is not SEQ ID NO: 1 and/or does not comprise a 10 amino acid sequence that is identical to a contiguous 10 amino acid sequence present in amino acids 108-128 of SEQ ID NO: 1.

Substitutes for an amino acid in the polypeptides of the invention are preferably conservative substitutions, which are selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can generally be substituted for another amino acid without substantially altering the structure of a polypeptide. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine, and histidine. The negatively charged (acidic) amino acids include aspartic acid and glutamic acid. Examples of preferred conservative substitutions include Lys for Arg and vice versa to maintain a positive charge; Glu for Asp and vice versa to maintain a negative charge; Ser for Thr so that a free —OH is maintained; and Gin for Asn to maintain a free NH2.

Other amino acids and derivatives thereof that can be used include 3-hydroxyproline, 4-hydroxyproline, homocysteine, 2-aminoadipic acid, 2-aminopimelic acid, -y-carboxyglutamic acid, (3-carboxyaspartic acid, ornithine, homoarginine, N-methyl lysine, dimethyl lysine, trimethyl lysine, 2,3-diaminopropionic acid, 2,4-diaminobutyric acid, homoarginine, sarcosine, hydroxylysine, substituted phenylalanines, norleucine, norvaline, 2-aminooctanoic acid, 2-aminoheptanoic acid, statine, (3-valine, naphthylalanines, substituted phenylalanines, tetrahydroisoquinoline-3-carboxylic acid, and halogenated tyrosines.

Polypeptide derivatives, as that term is used herein, also include modified polypeptides. Modifications of polypeptides of the invention include chemical and/or enzymatic derivatizations at one or more constituent amino acid, including side chain modifications, backbone modifications, and N- and C-terminal modifications including acetylation, pegylation, hydroxylation, methylation, amidation, and the attachment of carbohydrate or lipid moieties, cofactors, and the like.

Synthetic methods may be used to produce antimicrobial peptides, as is described in U.S. Pat. No. 6,486,125. Such methods are known and have been reported (Merrifield, Science, 85, 2149 (1963), Olson et al., Peptides, 9, 301, 307 (1988)). The solid phase peptide synthetic method is an established and widely used method which is described, for example, in the following references: Stewart et al., Solid Phase Peptide Synthesis, W. H. Freeman Co., San Francisco (1969); Merrifield, J. Am. Chem. Soc., 85 2149 (1963); Meienhofer in “Hormonal Proteins and Peptides,” ed.; C. H. Li, Vol. 2 (Academic Press, 1973), pp. 48-267; Bavaay and Merrifield, “The Peptides,” eds. E. Gross and F. Meienhofer, Vol. 2 (Academic Press, 1980) pp. 3-285; and Clark-Lewis et al., Meth. Enzymol., 287, 233 (1997). Peptides can be readily purified by fractionation on immunoaffinity or ion-exchange columns; ethanol precipitation; reverse phase HPLC; chromatography on silica or on an anion-exchange resin such as DEAF; chromatofocusing; SDS-PAGE; ammonium sulfate precipitation; gel filtration using, for example, Sephadex G-75; ligand affinity chromatography, and the like. Peptides can also be readily purified through binding of a fusion polypeptide to separation media, followed by cleavage of the fusion polypeptide to release a purified polypeptide.

The antimicrobial peptides may also be prepared via recombinant techniques well known to those skilled in the art. A polynucleotide sequence coding for an antimicrobial peptide can be constructed by techniques well known in the art. It will further be understood by those skilled in the art that owing to the degeneracy of the genetic code, a sizeable yet definite number of DNA sequences can be constructed to encode peptides having an amino acid sequence corresponding to a particular antimicrobial peptide. Once the DNA sequence has been determined, it can be readily synthesized using commercially available DNA synthesis technology. The DNA sequence can then be inserted into any one of many appropriate and commercially available DNA expression vectors through the use of appropriate restriction endonucleases. A variety of expression vectors useful for transforming prokaryotic and eukaryotic cells are well known in the art. The DNA sequences coding for the peptide are inserted in frame and operably linked to transcriptional and translational control regions, such as promoters, which are present in the vector and are functional in the host cell. The DNA sequence coding for the peptide can also be inserted into a system that results in the expression of a fusion protein that contains the antimicrobial peptide. For example, U.S. Pat. No. 5,595,887 describes methods of forming a variety of relatively small peptides through expression of a recombinant gene construct coding for a fusion protein that includes a binding protein and one or more copies of the desired target peptide. After expression, the fusion protein is isolated and cleaved using chemical and/or enzymatic methods to produce the desired target peptide.

### Administration and Formulation of Antimicrobial Peptides

The invention also provides pharmaceutical compositions that can be used for the administration of antimicrobial peptides of the invention to a subject in need thereof. In one example, a pharmaceutical composition can contain an RNase 7 peptide fragment as disclosed herein and a pharmaceutically acceptable carrier.

The pharmaceutical compositions of the invention may be prepared in many forms that include tablets, hard or soft gelatin capsules, aqueous solutions, suspensions, and liposomes and other slow-release formulations, such as shaped polymeric gels. An oral dosage form may be formulated such that the polypeptide or antibody is released into the intestine after passing through the stomach. Such formulations are described in U.S. Pat. No. 6,306,434 and in the references contained therein.

Oral liquid pharmaceutical compositions may be in the form of, for example, aqueous or oily suspensions, solutions, emulsions, syrups or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

The antimicrobial peptides can be formulated for parenteral administration (e.g., by injection, for example, bolus injection or continuous infusion) and may be presented in unit dosage form in ampules, prefilled syringes, small volume infusion containers or multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Pharmaceutical compositions suitable for rectal administration can be prepared as unit dose suppositories. Suitable carriers include saline solution and other materials commonly used in the art.

For administration by inhalation, antimicrobial peptides can be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, antimicrobial peptides may take the form of a dry powder composition, for example, a powder mix of a modulator and a suitable powder base such as lactose or starch. The powder composition may be presented in unit dosage form in, for example, capsules or cartridges or, e.g., gelatin or blister packs from which the powder may be administered with the aid of an inhalator or insufflator. For intra-nasal administration, antimicrobial peptides may be administered via a liquid spray, such as via a plastic bottle atomizer.

Antimicrobial peptides can be formulated for transdermal administration. Antimicrobial peptides can also be formulated as an aqueous solution, suspension or dispersion, an aqueous gel, a water-in-oil emulsion, or an oil-in-water emulsion. A transdermal formulation may also be prepared by encapsulation of a antimicrobial peptide within a polymer, such as those described in U.S. Pat. No. 6,365,146. The dosage form may be applied directly to the skin as a lotion, cream, salve, or through use of a patch. Examples of patches that may be used for transdermal administration are described in U.S. Pat. Nos. 5,560,922 and 5,788,983.

It will be appreciated that the amount of antimicrobial peptide required for use in treatment will vary not only with the particular carrier selected but also with the route of administration, the nature of the condition being treated and the age and condition of the patient. Ultimately the attendant health care provider may determine proper dosage. In addition, a pharmaceutical composition may be formulated as a single unit dosage form.

The antimicrobial peptides of this invention can be administered alone in a pharmaceutically acceptable carrier, as an antigen in association with another protein, such as an immunostimulatory protein or with a protein carrier such as, but not limited to, keyhole limpet hemocyanin (KLH), bovine serum albumin (BSA), ovalbumin, or the like. They may be employed in a monovalent state (i.e., free peptide or a single peptide fragment coupled to a carrier molecule). They may also be employed as conjugates having more than one (same or different) peptides bound to a single carrier molecule. The carrier may be a biological carrier molecule (e.g., a glycosaminoglycan, a proteoglycan, polyethylene glycol, albumin or the like) or a synthetic polymer (e.g., a polyalkyleneglycol or a synthetic chromatography support). Typically, ovalbumin, human serum albumin, other proteins, polyethylene glycol, or the like are employed as the carrier. Such modifications may increase the apparent affinity and/or change the stability of a peptide. The number of peptides associated with or bound to each carrier can vary, but from about 4 to 8 peptides per carrier molecule are typically obtained under standard coupling conditions.

It is further contemplated that the antimicrobial peptides disclosed herein may be used in combination with, or to enhance the activity of, other antimicrobial agents or antibiotics. In one embodiment a composition is provided comprising an antimicrobial peptides disclosed herein and a second antimicrobial agent. In one embodiment the second antimicrobial agent is an antibiotic. Combinations of antimicrobial peptides disclosed herein with other agents may be useful to allow antibiotics to be used at lower doses responsive to toxicity concerns, to enhance the activity of antibiotics whose efficacy has been reduced or to effectuate a synergism between the components such that the combination is more effective than the sum of the efficacy of either component independently.

In some embodiments, the antimicrobial agent is a quinolone antimicrobial agent, including for example but not limited to, ciprofloxacin, levofloxacin, and ofloxacin, gatifloxacin, norfloxacin, lomefloxacin, trovafloxacin, moxifloxacin, sparfloxacin, gemifloxacin, pazufloxacin or variants or analogues thereof. In some embodiments, the second antimicrobial agent is ofloxacin or variants or analogues thereof.

In some embodiments, the second antimicrobial agent is an aminoglycoside antimicrobial agent, including for example but not limited to, amikacin, gentamycin, tobramycin, netromycin, streptomycin, kanamycin, paromomycin, neomycin or variants or analogues thereof. In some embodiments, the second antimicrobial agent is gentamicin or variants or analogues thereof.

In some embodiments, the second antimicrobial agent is a beta-lactam antibiotic antimicrobial agent, including for example but not limited to, penicillin, ampicillin, penicillin derivatives, cephalosporins, monobactams, carbapenems, beta-lactamase inhibitors or variants or analogues thereof. In some embodiments, the second antimicrobial agent is ampicillin or variants or analogues thereof. In accordance with one embodiment the second antimicrobial agent is selected from a group consisting of penicillin, ampicillin, penicillin derivatives, cephalosporins, monobactams, carbapenems, or beta-lactamase inhibitors.

The compositions disclosed herein may include additional components that enhance their efficacy based on their desired use. In one embodiment the compositions are formulated as a pharmaceutical composition comprising any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents.

In one embodiment the antimicrobial peptide is coupled, bonded, bound, conjugated, or chemically-linked to one or more agents via linkers, polylinkers, or derivatized amino acids. In accordance with one embodiment the composition further comprises a lipid vesicle delivery vehicle. In one embodiment the lipid vesicle is a liposome or micelle. Suitable lipids for liposomal and/or micelle formulation include, without limitation, monoglycerides, diglycerides, sulfatides, lysolecithin, phospholipid, saponin, bile acids, and the like. The preparation of liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, the disclosures of which are incorporated herein by reference. In accordance with one embodiment a composition is provided comprising an antimicrobial peptide as disclosed herein and a lipid vesicle, wherein the peptide is encapsulated within the lipid vesicle, or linked to the surface of said lipid vesicle. In a further embodiment the composition may include additional active agents encapsulated or linked to the surface of the lipid vesicle delivery vehicle, including for example an anti-microbial agent such as an antibiotic. In one embodiment the lipid vesicle is a liposome, and in a further embodiment the liposome comprises an antimicrobial peptide linked to the exterior surface of the liposome. In one embodiment the antimicrobial peptides are covalently bound to the exterior surface of the liposome, optionally with additional active antimicrobial agents encapsulated within or linked to the exterior surface of the liposome.

Antibiotics suitable for use in accordance with the present description include for example, but are not limited to, a lantibiotic (e.g. nisin or epidermin), almecillin, amdinocillin, amikacin, amoxicillin, amphomycin, amphotericin B, ampicillin, azacitidine, azaserine, azithromycin, azlocillin, aztreonam; bacampicillin, bacitracin, benzyl penicilloyl-polylysine, bleomycin, candicidin, capreomycin, carbenicillin, cefaclor, cefadroxil, cefamandole, cefazo line, cefdinir, cefepime, cefixime, cefinenoxime, cefinetazole, cefodizime, cefonicid, cefoperazone, ceforanide, cefotaxime, cefotetan, cefotiam, cefoxitin, cefpiramide, cefpodoxime, cefprozil, cefsulodin, ceftazidime, ceftibuten, ceftizoxime, ceftriaxone, cefuroxime, cephacetrile, cephalexin, cephaloglycin, cephaloridine, cephalothin, cephapirin, cephradine, chloramphenicol, chlortetracycline, cilastatin, cinnamycin, ciprofloxacin, clarithromycin, clavulanic acid, clindamycin, clioquinol, cloxacillin, colistimethate, colistin, cyclacillin, cycloserine, cyclosporine, cyclo-(Leu-Pro), dactinomycin, dalbavancin, dalfopristin, daptomycin, daunorubicin, demeclocycline, detorubicin, dicloxacillin, dihydrostreptomycin, dirithromycin, doxorubicin, doxycycline, epirubicin, erythromycin, eveminomycin, floxacillin, fosfomycin, fusidic acid, gemifloxacin, gentamycin, gramicidin, griseofulvin, hetacillin, idarubicin, imipenem, iseganan, ivermectin, kanamycin, laspartomycin, linezo lid, linocomycin, loracarbef, magainin, meclocycline, meropenem, methacycline, methicillin, mezlocillin, minocycline, mitomycin, moenomycin, moxalactam, moxifloxacin, mycophenolic acid, nafcillin, natamycin, neomycin, netilmicin, niphimycin, nitrofurantoin, novobiocin, oleandomycin, oritavancin, oxacillin, oxytetracycline, paromomycin, penicillamine, penicillin G, penicillin V, phenethicillin, piperacillin, plicamycin, polymyxin B, pristinamycin, quinupristin, rifabutin, rifampin, rifamycin, rolitetracycline, sisomicin, spectrinomycin, streptomycin, streptozocin, sulbactam, sultamicillin, tacrolimus, tazobactam, teicoplanin, telithromycin, tetracycline, ticarcillin, tigecycline, tobramycin, troleandomycin, tunicamycin, tyrthricin, vancomycin, vidarabine, viomycin, virginiamycin, BMS-284,756, L-749,345, ER-35,786, S-4661, L-786,392, MC-02479, PepS, RP 59500, and TD-6424. In some embodiments, two or more antimicrobial agents (e.g., a composition comprising an antimicrobial agent and an antibiotic may be used together or sequentially. In some embodiments, another antibiotic may comprise bacteriocins, type A lantibiotics, type B lantibiotics, liposidomycins, mureidomycins, alanoylcholines, quinolines, eveminomycins, glycylcyclines, carbapenems, cephalosporins, streptogramins, oxazolidonones, tetracyclines, cyclothialidines, bioxalomycins, cationic peptides, and/or protegrins. In some embodiments, the antibiotic comprises one or more anti-anthrax agents (e.g., an antibiotic used in the art for treating B. anthracis (e.g., penicillin, ciprofloxacin, doxycycline, erythromycin, and vancomycin)).

In one embodiment a kit is provided for neutralizing pathogenic organisms. In one embodiment the kit comprises an antimicrobial peptide (as disclosed herein) and additional known antimicrobial agents, including one or more antibiotics.

The antimicrobial peptides can be conjugated to other polypeptides using standard methods such as activation of the carrier molecule with a heterobifunctional sulfosuccinimidyl 4-(n-maleimidomethyl)cyclohexane-1-carboxylate reagent. Cross-linking of an activated carrier to a peptide can occur by reaction of the maleimide group of the carrier with the sulthydryl group of a peptide containing a cysteine residue. Conjugates can be separated from free peptide through the use of gel filtration column chromatography or other methods known in the art.

For instance, peptide/carrier molecule conjugates may be prepared by treating a mixture of peptides and carrier molecules with a coupling agent, such as a carbodiimide. The coupling agent may activate a carboxyl group on either the peptide or the carrier molecule so that the carboxyl group can react with a nucleophile (e.g., an amino or hydroxyl group) on the other member of the peptide/carrier molecule, resulting in the covalent linkage of the peptide and the carrier molecule.

For example, conjugates of a peptide coupled to ovalbumin may be prepared by dissolving equal amounts of lyophilized peptide and ovalbumin in a small volume of water. In a second tube, 1-ethyl-3-(3-dimethylamino-propyl)-carboiimide hydrochloride (EDC; ten times the amount of peptide) is dissolved in a small amount of water. The EDC solution is added to the peptide/ovalbumin mixture and allowed to react for a number of hours. The mixture may then be dialyzed (e.g., into phosphate buffered saline) to obtain a purified solution of peptide/ovalbumin conjugate.

The amount of antimicrobial peptide that is delivered to the subject will depend upon the nature and severity of the condition being treated, and on the nature of prior treatments which the subject has undergone. Ultimately, the attending physician will decide the amount of antimicrobial peptide with which to treat each individual patient. Initially, the attending physician will administer low doses of polypeptide of the present invention and observe the patient's response. Larger doses of polypeptide of the present invention may be administered until the optimal therapeutic effect is obtained for the patient, and at that point the dosage is not increased further. It is contemplated that the various pharmaceutical compositions used to practice the method of the present invention should contain about 0.01 ng to about 100 mg (preferably about 0.1 μg to about 10 mg, more preferably about 0.1 μg to about 1 mg) of polypeptide of the present invention per kg body weight

Several illustrative embodiments of the invention are described by the following enumerated clauses:

1. An antimicrobial peptide comprising a contiguous fragment of SEQ ID NO: 1, wherein said fragment consists of

i) an amino acid sequence selected from amino acids 1-107 of SEQ ID NO: 1;

ii) a peptide having at least 90% amino acid sequence identity with SEQ ID NO: 5 or SEQ ID NO: 7; or

iii) a peptidomimetic derivative of i) or ii), with the proviso that the antimicrobial peptide does not comprise a 10 amino acid sequence that is identical to a contiguous 10 amino acid sequence present in amino acids 108-128 of SEQ ID NO: 1.

2. The antimicrobial peptide of clause 1 wherein said peptide has at least 90% or 95% sequence identity to an amino acid sequence of SEQ ID NO: 2, SEQ ID NO: 4, SEQ ID NO: 5, SEQ ID NO: 6 or SEQ ID NO: 7, or a peptidomimetic derivative of SEQ ID NO: 2, SEQ ID NO: 4, SEQ ID NO: 5, SEQ ID NO: 6 or SEQ ID NO: 7.

3. The antimicrobial peptide of clause 1 wherein said fragment consists of SEQ ID NO: 5 or SEQ ID NO: 7, or a peptide that differs from SEQ ID NO: 5 or SEQ ID NO: 7 by 1 to 8 amino acid substitutions.

4. The antimicrobial peptide of clause 3 wherein the amino acid substitutions are conservative amino acid substitutions.

5. A composition comprising the antimicrobial peptide of clause any one of clauses 1 to 4 and a pharmaceutically acceptable carrier.

6. The composition of clause 5 further comprising a supplemental anti-microbial agent.

7. A method for treating a subjecting having a bacterial infection by administering a therapeutically effective amount of the composition of clause 5 or 6 to the subject.

8. The method of any one clauses 5 to 7, wherein the subject is human.

9. The method of any one of clauses 5 to 8, wherein said composition comprises two or more RNase 7 peptide fragments selected from the group consisting of SEQ ID NO: 2, SEQ ID NO: 4, SEQ ID NO: 5 and SEQ ID NO: 6.

10. The method of any one of clauses 5 to 9, wherein the bacterial infection is caused by a gram-positive bacteria.

11. The method of any one of clauses 5 to 9, wherein the bacterial infection is caused by a gram-negative bacteria.

12. The antimicrobial peptide of any one of the preceding clauses wherein the peptide fragment of SEQ ID NO: 1 includes at least 45 amino acids.

13. The antimicrobial peptide of any one of the preceding clauses wherein the peptide fragment of SEQ ID NO: 1 includes at least 95 amino acids, but no more than 107 amino acids.

14. A method for treating or preventing a bacterial infection in a patient, said method comprising the step of,

administering to the patient an effective amount of a composition comprising the antimicrobial peptide of any one of clauses 1 to 4, 12 or 13.

15. The method of clause 14 wherein the infection is a urinary tract infection.

16. The method of clause 14 or 15 wherein the composition is administered on or in a biomedical device.

17. The method of any one of clauses 14 to 16 wherein the medical device is selected from the group consisting of staples, clips, drug delivery devices, stents, catheters, sutures, woven mesh, gauze, dressings, and growth matrices.

18. The method of any one of clauses 14 to 17 wherein the biomedical device is a selected from the group consisting of a catheter and a stent.

18. The method of any one of clauses 14 to 18 wherein the biomedical device is a catheter.

20. The method of clause 14 or 15 wherein the composition is administered directly into the bladder.

21. The method of any one of the preceding clauses wherein the infection is caused by an organism selected from the group consisting of Escherichia, Staphylococcus, Proteus Klebsiella, Enterococcus, Proteus, Morganella, Pseudomonas, Group B Streptococcus, and Candida.

22. The method of clause 21 wherein the organism is selected from the group consisting of E. coli, P. aeruginosa, K. pneumonia, P. mirabilis, E. faecalis, and S. saprophyticus.

23. The method of clause 22 wherein the organism is selected from E. coli, P. mirabilis, or S. saprophyticus.

24. The method of clause 23, wherein the bacterial infection is caused by Escherichia coli.

25. The method of clause 23, wherein the bacterial infection is caused by Staphylococcus saprophyticus infection.

26. The method of clause 23, wherein the bacterial infection is caused by Proteus mirabilis.

24. The method of any one of the preceding clauses, wherein the urinary tract infection is selected from the group consisting of cystitis, pyleonephritis, and urethritis.

25. A kit comprising a sterile vial, the antimicrobial peptide or composition of any one of the preceding clauses, and instructions for use describing use of the the antimicrobial peptide or composition for treating a urinary tract infection.

### EXAMPLES

The antimicrobial peptide Ribonuclease 7 (RNase7) is expressed in kidney and bladder epithelia. RNase7 plays a critical role in maintaining urinary tract sterility and possesses antimicrobial properties against gram-negative and gram-positive uropathogenic bacteria. The mechanisms of the antimicrobial activity of RNase7 involves in binding and depolarizing the bacterial membrane, a function that is different from those of the conventional antibiotics. RNase7 may serve as an interesting candidate for therapeutic agent for the bacterial infection. The goal of our study is to construct and find small peptides based on the sequences of RNase7 with higher antimicrobial activity and lower cytotoxicity activity.

The secondary structure of RNase7 consists of three α-Helixes and six β-strands. Therefore, a series of RNase7 fragments were constructed from both N and C-terminus of the full-length native peptide, starting with one that contains the first α-Helixes/β-strands and another that incorporates an additional α-Helixes/β-strands, etc. The sequences were generated by using PCR from full-length human RNase7 template and cloned into an E. coli expression vector pDEST-17, which adds an N-terminal His6 tag for affinity purification.

Ten fragments were expressed and purified, followed by testing against gram-negative uropathogenic E. coli (UT189). The Mean Inhibitory Concentration (MIC) or the concentration that kills 90% of the bacteria was determined for each fragment. The various fragments have different antimicrobial activity towards E. coli in which fragment as 1-97 has the strongest activity and fragments as 1-71 and 71-128 have the least activity. (See Table 1)

Further studies include testing against gram-positive bacteria and evaluation of their cytotoxic activity to calculate a therapeutic index (MIC/cytoxicity). Full length RNase 7 has enhanced activity against gram positives and no cytoxic or hemolytic properties.

### Methods

The tested bacterial strains were Escherichia coli (UTI89) and Staphylococcus Saprophyticus (ATCC 15305). Luria broth (LB) medium and Trypticase Soy Agar with 5% sheep blood were purchased from Fisher Scientific. Anti-RNase7 antibody was customized in rabbit with recombinant human RNase 7 (Biomatik).

### Plasmid DNA Construction

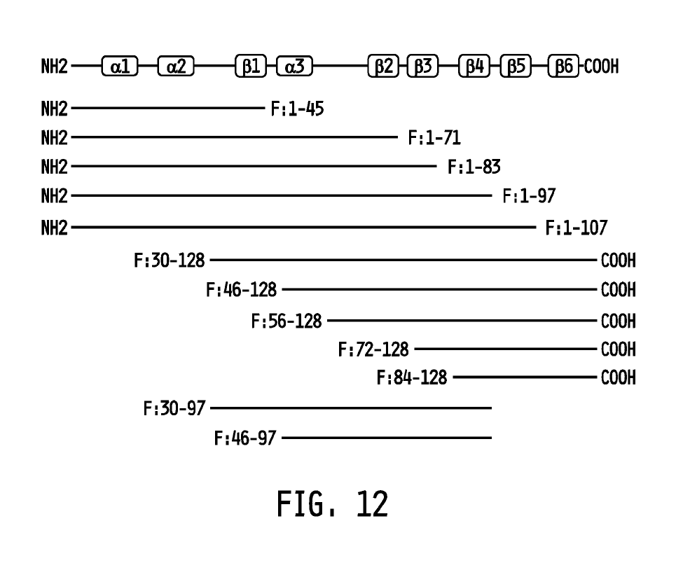

The full-length human RNASE7 sequence was generated from kidney cDNA library by PCR and cloned into E. coli expression vector pDEST17 (Invitrogen), which adds a six-residue histidine tag (His-tag) at the NH2-terminus. Ten fragmentary constructs of RNase7 were generated by PCR from full-length RNase7 template and cloned into vector pDEST17. Each contained different numbers of RNase 7 secondary structures starting from N-terminus or C-terminus of RNase7. Two expression vector that contained middle fragments were constructed as well (FIG. 12).

Each construct was transformed into E. coli BL21 Al (Invitrogen) to allow for L-arabinose inducible expression. Two-liter culture was grown to mid-log phase and peptide expression was induced by L-arabinose for 3 h. The cells were harvested and the pellets were resuspended in 30 ml of start buffer (20 mM sodium phosphate pH7.4, 500 mM NaCl, Phenylmethylsulphonyl fluoride (0.5 mM) and protease inhibitor cocktail) and lysed by sonication. Cell lysate was pelleted at 13000 r.p.m. for 20 minutes. The supernatant was applied to a Ni²⁺ charged HiTrap Chelating HP column (5 ml, GE). After washing with wash buffer (20 mM sodium phosphate pH7.4, 500 mM NaCl) containing 20 mM imidazole and 50 mM imidazole, respectively, the recombinant peptide was eluted with 500 mM imidazole. Purified peptides were dialyzed against DN (0) [25 mM Tris (pH 7.0), 0.1 mM EDTA, 10% glycerol] and the peptide concentrations were determined. 50 ng of each purified fragment was then analyzed by 18% SDS-PAGE to separate fragmentary peptides between 6 kD to 17 kD. The presence of each fragment was confirmed by western blot probed with anti-RNase7 antibody.

### Bacterial Viability Assay

Bacterial viability assays were performed using a Live/Dead BacLight™ bacterial viability kit (Invitrogen). Uropathogenic E. coli (UTI-89) and S. Saprophyticus (ATCC 15305) were grown at 37° C. to the mid-log phase, centrifuged at 5000 rpm for 5 min, and resuspended in water with an OD=0.2. Bacteria were stained using a 1:1 mixture of a green-fluorescent STY®9 dye and a red-fluorescent propidium iodide dye as provided in the Live/Dead BacLight™ bacterial viability kit. SYTO®9 labels both live and dead bacteria. Propidium iodide penetrates only bacteria with damaged membranes. Consequently, live bacteria with intact membranes fluoresce green, while dead bacteria with damaged membranes fluoresce red. 25. 50 uL of stained uropathogenic bacteria was mixed with recombinant RNase 7 or RNase 7 fragments to achieve a final concentration of 3 uM and 1 uM. 10 uL of this mixture was added to poly-1-lysine coated microscope slides (Polysciences, Inc, Warrington, Pa., USA) and confocal images of the bacteria were obtained using a Zeiss LSM 700 confocal laser scanning microscope (Carl Zeiss LLC, Thornwood, N.Y., USA). SYTO®9 was excited using an argon laser (448-500 nm emission collected) and propidium iodide was excited using an orange diode (555-600 nm emission collected). Samples were imaged in triplicate.

### Antimicrobial Activity Assay

The antimicrobial activity of RNase 7 and its fragments was estimated using a microdilution assay. Briefly, test bacteria (10⁵) were incubated with several concentrations of RNase 7 and each fragment in 50 μl 0.1×PBS buffer for 3 hours in a 37° C. incubator. Each reaction was plated on the appropriate medium and grew overnight at 37° C. The antimicrobial activity was determined by the number of colony-forming units (CFUs) the following day. The minimum inhibition concentration (MIC) is defined as the lowest concentration that inhibits the growth of 90% of the inoculation and the minimum bactericidal concentration (MB C) is defined as the lowest concentration at which less than 99.99% of the initial inoculum is viable.

### Binding of RNase7 Fragments to Bacteria

Recombinant RNase7 and its fragments were each incubated with bacteria (5*10⁶ cfu) in 50 μL volume with a final protein concentration at 1.5 μM for 1 hour at 37° C. the bacteria were then spun down and washed with 10 mM sodium phosphate two times. The inputs, supernatants and pellets were analyzed by SDS-PAGE and visualized by silver staining and western blot.

Assays of the permeabilization of bacterial membrane were performed with SYTOX green nucleic acid stain (Invitrogen™). The overnight cultures of bacteria were centrifuged at 5000 rpm for 5 min, the pellets were then washed with water and resuspended in water with an OD=0.5. 90 μl of each bacteria were then incubated with 10 μl of peptides with different concentrations for 3 hours at 37° C. in a 96-well plate. 100 μl of 2 μM SYTOX Green (Invitrogen™) were then added to each well and incubated in dark for 15 min. SYTOX green penetrates cells with compromised membranes not the membranes of live cells. The increase of fluorescence was measured using 485 and 520-μm filters for excitation and emission wavelengths, respectively. Bacteria alone and bacteria treated with 70% ethanol were used as controls and the fluorescence from each was recorded as Fmin and Fmax. The percentage of cell death at each concentration for each fragment was calculated as below: death %=(F−Fmin)/(Finax−Fmin)*100%

Recombinant RNase 7 presents potent antimicrobial activity against both gram-positive and gram-negative uropathogenic bacteria.

Described herein is the antimicrobial activity of recombinant RNase 7 against uropathogenic gram-negative E. coli, UTI89 and gram-positive S. Saprophyticus, ATCC 15305. The MIC of full-length RNase 7 is 0.26 LM for E. coli and 0.22 uM for S. Saprophyticus. These results are consistent with previous reports[1, 2] that RNase 7 presents potent antimicrobial activity against both strains with an MIC in mircomolar range.

Described herein is minimum inhibitory concentrations (MICs) and minimum bactericidal concentrations (MBCs) of RNase7 C-terminal fragments. The C-terminal fragments present gradually decreased activity against B. coli when the N-terminus of RNase 7 was removed suggested by both MICs and MBCs. When the first 71 amino acids were removed, the resulting peptide had lowered antimicrobial activity. When tested against S. Saprophyticus, the MIC of each fragment was relatively unchanged until the first 71 amino acids were removed. However, the MBCs of the peptide fragments were significantly affected when the first 30 amino acids were removed.

Described herein is a permeabilization assay to study how these fragments penetrate the microbial membrane. In this assay SYTOX green was used to detect the presence of the dead cells with compromised plasma membranes. All the fragments show decreased activity against both E. coli and S. Saprophyticus. Without being bound by theory, it is believed that this result suggests that the N-terminus of RNase 7 is important for the protein to penetrate the bacterial membrane for both stains.

N-terminal fragments of RNase 7 present relatively unchanged activities against both E. coli and S. Saprophyticus reflected by MICs and MBCs except two fragments. Fragment 1-71 showed significantly decreased activity against B. coli but not S. Saprophyticus while fragment 1-97 presented increased activity against both strains.

The permeability of each N-terminal fragment was also evaluated. F:1-97 shows increased permeability and fragment F:1-71 shows permeability against S. Saprophyticus but lower permeability against E. coli. Others

Fragment 1-97 has increased activity against both gram-positive and gram-negative bacteria. Described herein is a fragment presenting enhanced activity against both strains. Compared with full-length RNase 7, the MIC and MBC of F:1-97 against E. coli and S. saprophyticus is decreased relative to the full-length peptide (Table 1). In a Bacterial Viability Assay, tested bacteria were incubated with full-length RNase 7 and fragment F:1-97 with a final concentration at 1 μM for 30 min and 3 hours. Live cells were labeled with a green fluorescent dye and dead cells were labeled with a red fluorescent dye. Within 30 min, F:1-97 killed most of the E. coli and S. saprophyticus while full-length RNase 7 did not. After 3 hours, all the E. coli and S. saprophyticus were completely dead.

F:1-71 shows antimicrobial activity against S. saprophyticus but lower activity against E. coli.

F:1-71 has a reduced activity with an MIC 1.93 μM and MBC 3.86 μM against E. coli. However, its antimicrobial activity against S. saprophyticus is similar to other N-terminal fragments. As for F:1-97 bacteria were incubated with F:1-71 with a final concentration at 3 μM for 30 min and 3 hours.

### AMP Structure and Antimicrobial Activity:

Associations between AMP structure and antimicrobial activity have been reported Changes in sequence, composition, or intramolecular bonds may affect the structure-function relationships between antimicrobial peptides and bacteria. In some AMPs, a single amino acid substitution can significantly alter function.

### Ribonuclease 7 (RNase7):

RNase 7 is a 14.5 kDa AMP that is arranged 3 α-helices and 2 triple-stranded, antiparallel β-sheets. Only limited information is known on its structure-function relationship. It has been previously described that 22 positively charged residues (Lys₁₈ and Arg₄), distributed in 3 clusters, are important for membrane disruption activity. Although the mechanisms for RNase 7's antimicrobial properties are not completely understood, its bactericidal activity has been linked to its capacity to permeate and disrupt the bacterial membrane, independent of its ribonuclease activity. Consequently, RNase 7 has potent antimicrobial activity against gram-negative bacteria, gram-positive positive bacteria, and yeast. It has been stated that, on a per molar base, RNase 7 is one of the most potent human AMPs. Its mean inhibitory concentration (MIC) is usually in the low micromolar range even for highly resistant bacteria such as vancomycin-resistant Enterococcus faecalis (E. faecalis). Harder and Schröder first identified RNase 7 as an abundant protein in the human epidermis while examining protein extracts of normal skin for antimicrobial activity. Subsequent studies demonstrated that RNASE7 is expressed in other organs, including the liver, gastrointestinal tract, heart, skeletal muscle, and respiratory tract. RNASE7 expression was noted in the kidney, although the extent of its expression and precise location were not characterized.

### RNase 7 in the Kidney and Urinary Tract:

The expression and relevance of RNase 7 in the human kidney and urinary tract has been previously described. Using RNA isolated from healthy human tissue, quantitative real-time PCR has previously shown that basal RNASE7 expression in kidney and bladder tissue. Immunostaining localized RNase 7 protein to the urothelium of the bladder, ureter, and intercalated cells of the collecting tubules. In healthy control human urine samples, 5.6-20.0 μg RNase 7 per mg creatinine is detected, which corresponds to a concentration of 0.15-0.30 μM. Previously described antibacterial neutralization assays showed that urinary RNase 7 has potent antimicrobial properties against gram-negative and gram-positive uropathogenic bacteria.

Cloning Human RNase 7 for Recombinant Protein Production:

The reading frame of RNase 7 amino acids 1-128 was PCR amplified from a cDNA library extracted from human kidney. The amplified RNASE7 sequences were then transferred into the Escherichia Coli (E. coli) expression vector pDEST-17 (Invitrogen, Carlsbad, Calif.) to add an NH₂-terminal His tag for protein isolation. Standard DNA sequencing verified plasmid insert and correct nucleotide sequences. Nickel-chelating chromatography was used to affinity purify recombinant RNase 7.

Development of Recombinant Human RNase 7 Fragments:

Described herein are expression clones constructed to produce RNase 7 fragments. The fragments were amplified by PCR using full length RNASE7 as the template. Primers were designed to be complementary to the sequences of each end of the fragment and flanked by the attB sequences that are required for cloning into the expression vector. The PCR products were purified and cloned into pDEST17 plasmids.

RNase7 Antibacterial Activity:

Laboratory bacterial strains from the American Type Culture Collection (ATCC, Manassas, Va.) and strains isolated from positive urine cultures were obtained from the Microbiology Laboratory at Nationwide Children's Hospital. The mean inhibitory concentrations (MIC) of RNase 7 for ATCC non-uropathogenic strains (N=3 distinct strains) were 1.3 μM while our uropathogenic E. coli isolated (N=3) had a MIC of 2.5 μM.

The smallest RNase 7 fragments from the N-terminal and C-terminal ends contain 2 α helices and 1 β sheet, respectively. With progressively increased fragment size, more α helices and/or β sheets are incorporated.

Fragment MIC:

RNase 7 F:1-71 (FIG. 12) had a MIC of 0.63 μM against a uropathogenic E. coli, which was lower than the 2.5 μM MIC for full length RNase 7.

### Atomic Force Microscopy (AFM) and Bacterial Morphology

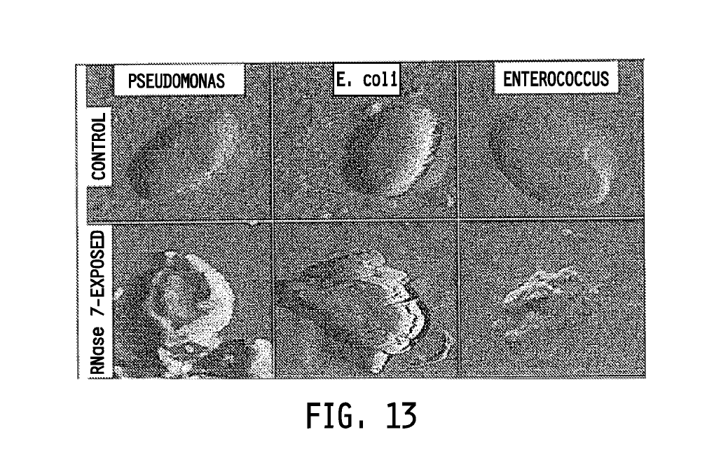

The high-resolution AMF images demonstrate nanometer-scale changes in cell morphology including pores, spore rodlets and bacterial division septa. (See FIG. 13) 1×10⁹ cfu of E. Coli, P. aeruginosa and E. faecalis were incubated with twice the MIC of RNase 7. Ultra-pure water was used as a negative control. AFM imaging was performed with the Multimode AFM instrument (Veeco, Santa Barbara, Calif.) at Ohio State. Differences between control and bacteria exposed to RNase 7 are demonstrated in FIG. 13. This demonstrates that RNase 7 has direct bacteriocidal effects on multiple microbes.

### Determine Eukaryotic Cell Toxicity:

AMP toxicity against human cells can diminish the therapeutic potential of AMPs. Therefore quantification of cytotoxicity and hemolytic profiles is an important objective of AMP related research.

Hemolytic assay: To determine the hemolytic activity of RNase 7, washed human erythocytes are incubated for 1 hour at 37° C. with a serial two-fold dilution of recombinant RNase 7 and its fragments in pyrogen free saline. After incubation, centrifugation to remove intact erythrocytes and a 10-fold dilution of supernatant, the concentration of released hemoglobin is measured in a micotitre plate reader at 405 nm. (Biochem J. Feb. 15 2008; 410(1):113-122) The peptide concentration that results in 50% hemolysis of erythrocytes will be designated the HC50.

Cytotoxic assay: The urothelial cells that line the bladder and distal nephron epithelial cells are the primary areas of RNase7 protein expression in the kidney and urinary tract. Thus, the cytotoxicity of variant peptides on these epithelia is determined. Human primary bladder urothelial cells (Catalog #4320, ScienCell Research Laboratories, Carlsbad, Calif.) and renal medullary epithelial cells (Catalog # FC-0018, Lifeline Cell Technology, Frederick, Md.) are cultured according to the manufacture's instructions. Cytotoxicity is measured with the Live/Dead Viability/Cytotoxicity Kit for mammalian cells (Catalog number # L3224, Invitrogen/Molecular Probes, Eugene Oreg.) according to the manufacturer's fluorescence microplate protocol and expressed as the percentage of viable cells after treatment with serial two-fold dilutions of RNase 7 and its deletion fragments incubated for 1 hour at 37° C. The results of the cytotoxic assay are reported as the EC50 or the concentration that results in 50% cell death.

Therapeutic index: The ability of RNase 7 and its deletion fragments to kill bacteria with limited toxicity to human cells will be determined by calculating the therapeutic indices (HC50/MIC and EC50/MIC). (53) Thus, a larger therapeutic index demonstrates greater specificity for bacterial cells.

Statistical Quantitative structure activity relationship (QSAR) modeling is performed on the RNase 7 fragments using JMP and SAS software packages (SAS Institute Inc., Cary, N.C.). RNase 7 fragment properties to be correlated with the MIC, HC50 and EC50 include charge, mean hydrophobic moment, maximum hydrophobic moment, hydrophobicity, conformation/secondary structures, amino acid ratios and sequence length. The net charge, hydrophobicity and secondary structure propensity are calculated with the Collection of Antimicrobial Peptides Comprehensive Antimicrobial Peptide Database AMP tool. The hydrophobic moment is calculated with H-moment software (Sanger Centre, Cambridge UK). Variable selection will use the all-possible-models method in SAS. Evaluate bacterial membrane disruption:

RNase 7 treatment of bacteria: Bacteria are cultured, centrifuged and re-suspended in ultra-pure water to a final concentration of 1×10⁸ cells/ml and mixed with an equal volume of recombinant RNase 7 and RNase 7 deletion fragments. The recombinant RNase 7 and RNase 7 fragment (FIG. 12) concentrations will be in increments of 25%, 50%, and 100% of the LD50 for each specific pathogen. (35) Ultra-pure water is used as a negative control. Bacteria are exposed to RNase 7 or negative control for 5-minute increments up to 1 hour. Bacteria are prepared in a variety of conditions, including live versus fixed.

AFM imaging: It is believed that small variations in AMP structure can lead to changes in antimicrobial activity. These changes can alter the MIC It has be previously described that RNase 7 has 3 clusters of cationic lysine residues on its surface and that an amino acid substitution in one of these lysine clusters significantly reduces its antimicrobial activity.

Site-directed mutagenesis: The full length RNase 7 plasmid described herein is used to make the following changes. RNase 7 variants are generated from this plasmid using site-directed mutagenesis (QuickChange, Catalog #200519, Stratagene, Santa Clara, Calif.). Variant sequences will be confirmed by DNA sequencing.

TABLE A

| Desired Change | AA substitution | # of target AA |
|---|---|---|
| Increase hydrophobicity | Ala changed to Leu | 12 |
| Decrease hydrophobicity | Leu changed to Ala | 15 |
| Increase net charge | Ser changed to Lys | 12 |
| Decrease net charge | Lys changed to Ser | 18 |

Targets: The role of net charge and hydrophobicity on RNase 7's mechanism of action is made systematically decreasing or increasing these parameters (Table A). Disulfide bonds between cysteine residues have been linked to antimicrobial activity. RNase 7 contains 8 cysteine residues connected through 4 disulfide bridges. It is believed that these disulfide bonds stabilize the overall peptide structure. Cys residues are systematically exchanged to disrupt these bonds by mutating a cysteine to a serine. This substitution does not alter the hydrophobicity or charge of the peptide as the —SH side-chain is substituted to an —OH side chain. Other changes include substitution of lysine with glutamine to change net charge.

Ribonuclease 7 (RNase 7) is a 14.5 kDa peptide that possesses potent antimicrobial properties against Gram-negative and Gram-positive bacteria and is expressed in a variety of epithelial tissues. Little is known about its mechanisms of action and the determinants of its antimicrobial properties. Described herein is a series of RNase 7 fragments that were prepared and that contained different components of its secondary motifs starting from both the N-terminus and C-terminus of RNase 7. the antimicrobial properties of each fragment against both Gram-positive S. saprophyticus and Gram-negative E. coli and P. mirabilis are described. RNase 7 fragments displayed significant differences in their antimicrobial activity profiles. Compared to N-terminal fragments, C-terminal fragments showed uniformly decreased activity against Gram-negative E. coli, P. mirabilis, and Gram-positive S. saprophyticus. Fragments that lack β-sheet 1, 3 and 4 also demonstrated decreased activities. Described herein is a fragment with at least four-fold increased potency against both E. coli and Staphylococcus compared to full-length peptide. Described hereinare distinct regions of the peptide that affect Gram-negative and Gram-positive activity. The results described herein suggest that distinct mechanisms may be responsible for RNase 7's antimicrobial activity against various uropathogens.

Antimicrobial peptides (AMPs) are small proteins that possess antimicrobial activity against bacteria, enveloped viruses, fungi, and some protozoa. AMPs are usually cationic secondary to the presence of lysine and/or arginine residues and amphipathic, which allows them to intercalate into hydrophobic bacterial cell membranes yet remain in solution in aqueous environments. Because pathogenic bacteria are still susceptible to endogenous AMPs, they have been considered a possible treatment against drug resistant organisms.

Unfortunately, our understanding of the fundamental mechanisms of AMP action and structure-functional relationship with bacteria is cursory. The associations between AMP structure/properties and antimicrobial activity have been reported include:

1. Net charge: The net charge refers to the entire molecular charge. A higher positive net charge, increases electrostatic attraction to negatively charged bacterial cell membranes (18).

2. Amphipathicity: Amphipathicity refers to the ratio of polar and nonpolar components. Increased amphipathicity as estimated by the hydrophobic moment allows for solubility in pathogen membranes (8).

3. Hydrophobocity: Hydrophobicity quantitates the percentage of hydrophobic residues within a peptide. The ability of an AMP to intercalate into a bacterial membrane increases as hydrophobicity increases (18).

4. Conformation: The dimensional topography of a peptide can influence activity. The majority of AMPs have an α-helical and/or β-sheet conformation (18).

Ribonuclease 7 (RNase 7) is a 14.5 kDa AMP, which Harder and Schroder first identified as an abundant protein in the human epidermis while examining protein extracts of normal skin for antimicrobial activity. Subsequent studies demonstrated that RNASE7 is expressed in other organs, including the liver, gastrointestinal tract, heart, skeletal muscle, and respiratory tract. This antimicrobial peptide has potent activity against gram-negative bacteria, gram-positive positive bacteria, and yeast. The mature peptide is arranged as 3 α-helices and 2 triple-stranded, antiparallel β-sheets. Although the mechanisms for RNase 7's antimicrobial properties are not completely understood, its bactericidal activity has been linked to its capacity to permeate and disrupt the bacterial membrane, independent of its ribonuclease activity. Of note, the flexible coil at the N-terminus that contains 2 lysine residues have been shown to be critical for membrane disruption.

The expression and relevance of RNase 7 in the human kidney and urinary tract has been previously described. Antibacterial neutralization assays showed that urinary RNase 7 has potent antimicrobial properties against Gram-negative and Gram-positive uropathogenic bacteria. Generation of AMP fragments that contain distinct secondary structures such as α-helices has been used to gain insight into the structure-function activity relationship of other AMPs. The objective of this study is to generate RNAse 7 fragments to identify the intrinsic functional domains of RNase 7 that influence its activity on uropathogenic E. coli, S. saprophyticus, and P. mirabilis.

### Additional Methods:

Bacterial Strains and Antibody:

Uropathogenic Escherichia coli (UTI89) was grown in Luria broth (LB) medium and plated on LB-agar. Uropathogenic Staphylococcus saprophyticus (ATCC 15305) was grown in LB and plated on Trypticase Soy Agar with 5% sheep blood (Fisher Scientific, Pittsburgh, Pa., USA). Uropathogenic Proteus mirabilis (ATCC 7002) was grown in LB and plated on MacConkey II Agar (Fisher Scientific, Pittsburgh, Pa., USA). These organisms were chosen as the primary organisms that account for a majority of Gram-negative and Gram-positive urinary tract infections (UTI). Anti-RNase 7 antibody was generated in rabbit with recombinant human RNase 7 (Biomatik, Wilmington, Del., USA)

Plasmid DNA Construction:

The full-length human RNASE7 sequence was generated from kidney cDNA library by PCR and cloned into E. coli expression vector pDEST17 (Invitrogen, Carlesbad, Calif., USA), which adds a six-residue histidine tag at the N-terminus. Ten fragmentary constructs of RNase 7 were generated by PCR from full-length RNase 7 template and cloned into vector pDEST17. Each contained different numbers of RNase 7 secondary structures starting from N-terminus or C-terminus of RNase 7. Two expression vectors that contained middle fragments (neither the N- or C-terminus) were constructed as well (FIG. 12). The sequences of each construct were confirmed by DNA sequencing.

Recombinant Peptide Expression and Purification:

Recombinant peptides were expressed and purified as described previously (Koten, et al., PloS one 4:e6424). Briefly, each construct was transformed into E. coli BL21 AI (Invitrogen) to allow for L-arabinose inducible expression. Bacteria were grown in 2 L cultures to mid-log phase and peptide expression was induced by L-arabinose for 3 hours. The cells were harvested, and the pellets were resuspended in 30 ml of start buffer (20 mM sodium phosphate pH 7.4, 500 mM NaCl, 0.5 mM Phenylmethylsulphonyl fluoride and protease inhibitor cocktail (Sigma, St. Louis, Mo., USA) and lysed by sonication. Cell lysate was pelleted at 13000 rpm for 20 min. The supernatant was applied to a Ni²⁺ charged HiTrap Chelating HP column (General Electric, Piscataway, N.J., USA). After washing with wash buffer (20 mM NaPO₄ pH 7.4, 500 mM NaCl) containing 20 mM imidazole and then 50 mM imidazole, the recombinant peptide was eluted with 500 mM imidazole. Purified peptides were dialyzed against DN (0) buffer [25 mM Tris (pH 7.0), 0.1 mM EDTA, 10% glycerol], and peptide concentrations were determined with a Bradford protein assay (Bio-Rad, Hercules, Calif., USA) and confirmed with Pierce BCA assay (Pierce, Rockford, Ill., USA). The presence of each fragment was subjected to SDS-PAGE and identified by immunoblot using an anti-RNase7 antibody at 1:2000 titer.

Antimicrobial Kill Assay:

Antimicrobial activities of the recombinant peptides were estimated with a microdilution assay. Briefly, the test bacteria (10⁴ CFUs) were incubated with various concentrations of RNase 7 and each fragment in 50 ul 0.1×PBS buffer for 3 hours at 37° C. After incubation on Agar plates overnight at 37° C., the number of colony-forming units (CFUs) at each peptide concentration was determined. Using untreated bacterial aliquots as baseline, the minimum inhibition concentration (MIC) was defined as the lowest concentration that inhibits the growth of 90% of the inoculation, and the minimum bactericidal concentration (MBC) was defined as the lowest concentration at which less than 99.99% of the initial inoculum is viable.

Bacterial Viability Assay:

To confirm the accuracy of our MIC/MBC data, cell death assays using SYTOX Green (Invitrogen), which selectivity labels nucleic acids of cells with compromised membranes of dead/dying cells were performed. Overnight cultures of bacteria were centrifuged at 5000 rpm for 5 minutes. Pellets were then washed with PBS (pH 7.4) and resuspended in PBS to an OD₆₀₀ of 0.5 for Escherichia coli (UTI89) and OD₆₀₀ of 0.8 for Staphylococcus saprophyticus. Per manufacturerer's instructions, 90 μl of each bacteria strain were incubated with 100 μl of 2 uM SYTOX Green for 15 minutes. Ten microliters of peptides at various concentrations were added for 3 hours at 37° C. in a 96-well plate. Compromised bacterial membranes of dead/dying cells will be labeled while lives cells are not labeled as noted by Invitrogen. Fluorescence was measured using 485 and 520-nm filters for excitation and emission wavelengths, respectively. Untreated bacteria were used as a 100% live control, and fluorescence was noted as Fₘᵢₙ. To elicit complete killing, bacteria were treated with 70% ethanol for 1 hour. This experimental group's fluorescence was noted as Fₘₐₓ. The percentage of cell death at each concentration for each fragment was calculated as (F_{fragment}−Fₘᵢₙ)/(Fₘₐₓ−Fₘᵢₙ)×100%.

For images of intact versus disrupted membranes of dying/dead cells, the Live/Dead BacLight™ bacterial viability kit (Invitrogen) was used. E. coli and S. saprophyticus were grown at 37° C. to the mid-log phase, centrifuged at 5000 rpm for 5 min, and resuspended in water to an OD₆₀₀ of 0.2. Bacteria were incubated in a 1:1 mixture of Live/Dead BacLight™ bacterial viability assay. A bacterial aliquot of 50 μL was mixed with recombinant RNase 7 or recombinant fragments to achieve a final concentration of 1 μM. Ten microliters of this mixture was added to poly-1-lysine coated microscope slides (Polysciences, Inc, Warrington, Pa., USA), and images were obtained in triplicate using a Zeiss LSM 710 confocal laser scanning microscope (Carl Zeiss LLC, Thornwood, N.Y., USA).

Urinary Condition Functional Assays:

Because the urinary environment can have significant variation in salt concentration, ionic composition, and pH, an antimicrobial/bacteriostatic assay was performed to evaluate if pH, calcium, or ionic strength affects the activity of full-length RNase7 or its fragments. The highest activity fragment F:1-97 and lowest activity fragment F:72-128 for these experiments were chosen. Bacteria were inoculated in 1% peptone water and growth to log phase with OD₆₀₀=0.5. Bacteria were diluted to 1:10 and 1 μl added to 100 μl of 1% peptone water with various pH, CaCl₂ or NaCl concentrations that correspond to a physiologic range present in the urine in a 96-well flat bottom plate (Thermo Scientific, Nunc, Worcester, Mass.). To each well, 2 μM of RNase7 or its fragments were added.

Bacterial growth was monitored using an Absorbance microplate reader (BioTek Instruments, Winooski, Vt.) at a final volume of 110 μl. The turbidity of the culture was measured and recorded at t=0 and every 20 min thereafter for 10 hours using the absorbance at 600 nm (OD₆₀₀). The values of OD₆₀₀ values were plotted against times to generate growth curves.

Circular Dichroism (CD) Spectroscopy:

To determine if recombinant RNase 7 peptides retained secondary structure, CD experiments were performed by The Protein Facility at Iowa State University. Briefly, using 0.5 mm path length cuvettes with 0.5 mg/ml of each peptide in 10 mM Tris-HCl pH 7.6, the CD spectra for the full-length RNase7, F:1-97, F:56-128 and F:72-128 were recorded from 190 nm to 250 nm using a wavelength step of 0.1 nm. The curves were fitted and analyzed using CD-FIT program. All peptide fragments demonstrated retention of some secondary structure with alpha helices and beta sheets present in each peptide.

Statistical Analyses:

To determine if the inhibitory (MIC) and killing (MBC) values presented in Table 1 had statistically significant difference compared to full-length peptide, all raw data was subjected to statistical analysis. Briefly, time-to-event analysis was performed for each peptide fragment for each MIC and MBC data point. Chi-square values with Bonferroni adjustments to p-values to account for multiple testing for each fragment were determined using SAS software (SAS Institute, Cary, N.C., USA). For all fragments, p-values less than 0.05 were considered statistically significant.

Fragment AMP Property Determination:

To determine if individual amino acid properties were associated with fragment antimicrobial activity, the net charge and hydrophobicity were calculated with the Collection of Antimicrobial Peptides Comprehensive Antimicrobial Peptide Database AMP tool. The mean and maximum hydrophobic moment, a measure of amphipathicity, was calculated with H-moment software (Sanger Centre, Cambridge, UK).

Recombinant RNase 7 has Potent Antimicrobial Activity:

Antimicrobial activity of recombinant RNase 7 was determined against uropathogenic Gram-negative E. coli and P. mirabilis, and uropathogenic Gram-positive S. saprophyticus (Table 1), The MIC of full-length RNase 7 was 0.26 μM for 0.88 μM for P. mirabilis and 0.22 μM for S. saprophyticus. The results are consistent with previous reports that demonstrates RNase 7 possesses potent antimicrobial activity against multiple uropathogenic bacterial species at micromolar concentrations.

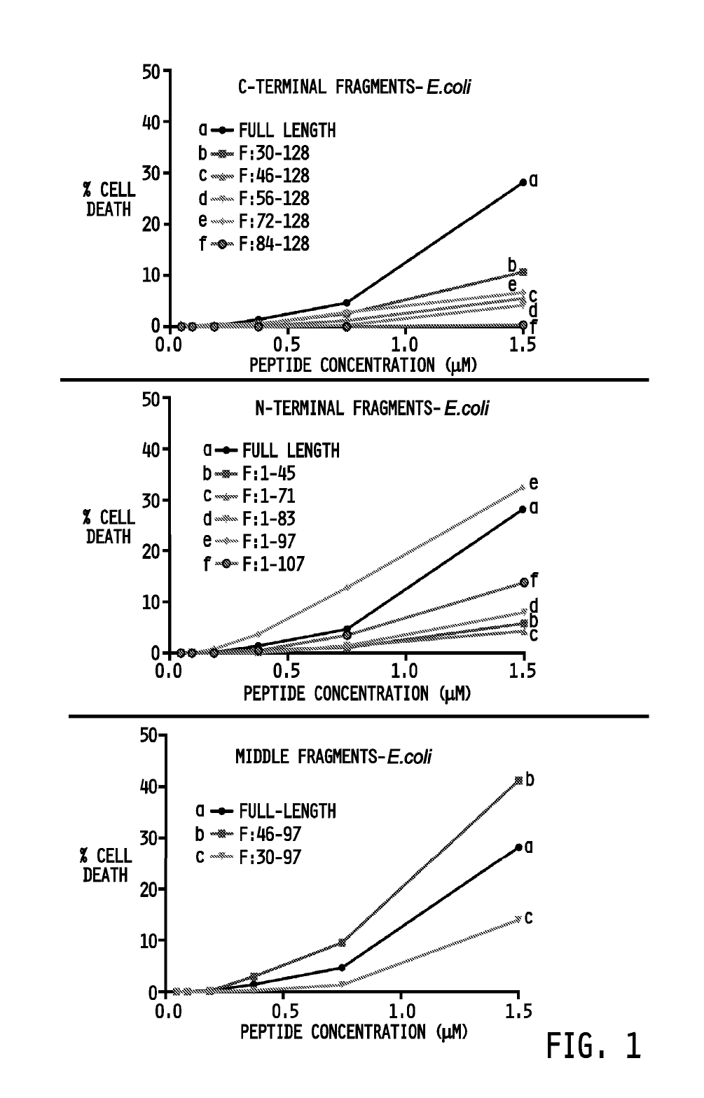

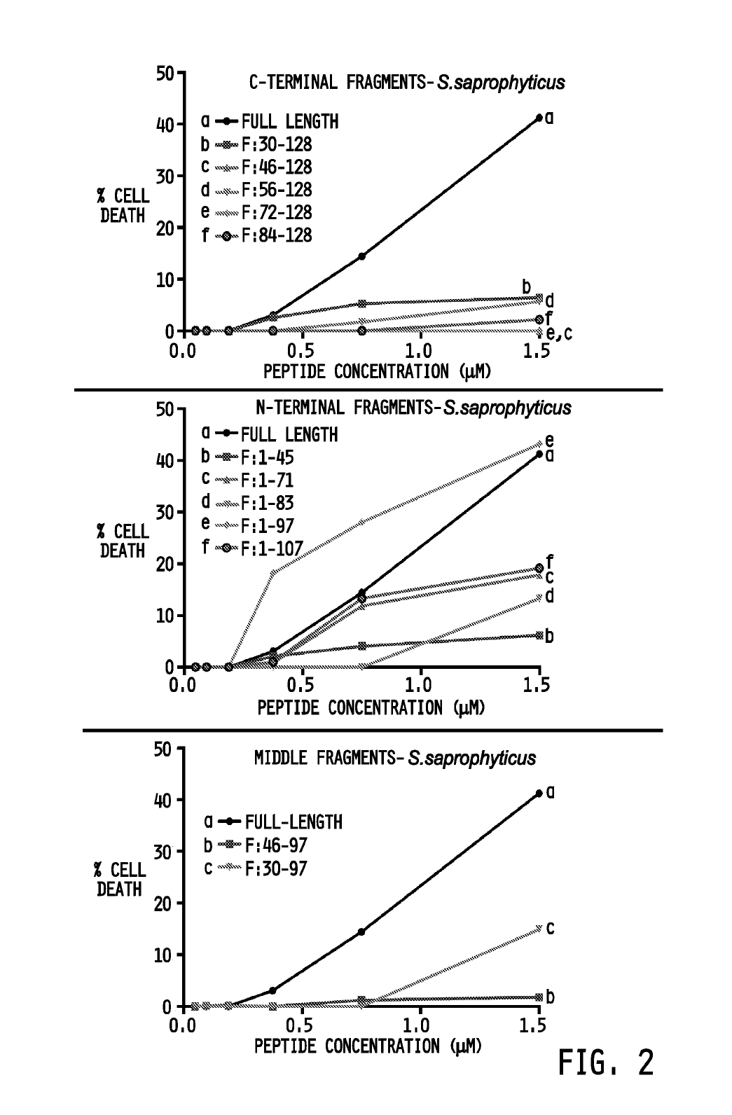

The C-terminal fragments generally had activity that was reduced compared to full-length RNase 7 (Table 1). Two fragments, F:46-128 and F:56-128, had MICs similar to full-length against S. saprophyticus. Fragments devoid of the initial 71 amino acids (F:72-128 and F:84-128) demonstrated markedly diminished activity. The MIC/MBCs of these fragments were 4 to 16-fold higher than the full-length protein against E. coli and S. saprophyticus (Table 1). None of the C-terminal fragments had activity against P. mirabilis (Table 1). Furthermore, C-terminal fragments had decreased E. coli and S. saprophyticus membrane permeabilization and subsequent death compared to full-length peptide (FIG. 1 and FIG. 2).

Compared to the C-terminal fragments, the N-terminal fragments had greater antimicrobial activity. The N-terminal fragments have a range of activity against E. coli and S. saprophyticus compared to full-length protein (Table 1). Generally, fragments showed variable activity compared to full-length protein with most demonstrating a statistically insignificant altered activity against E. coli and S. saprophyticus (Table 1). Interestingly, F:1-97 had significantly increased activity against E. coli and S. saprophyticus with MICs and MBCs that were at least 4-fold lower than full length protein. Conversely, the F:1-71 fragment had dramatically decreased activity against E. coli with a seven-fold higher MIC and MBC compared to full length protein. Of note, F:1-71 had the same activity to S. saprophyticus as full length protein.

TABLE 1
Fragment MICs and MBCs

| | E. coli | | S. saprophyticus | | P. mirabilis | |
|---|---|---|---|---|---|---|
| | MIC | MBC | MIC | MBC | MIC | MBC |
| Peptide | (uM) | (uM) | (uM) | (uM) | (uM) | (uM) |
| RNase7 | 0.26 | 0.53 | 0.22 | .44 | .88 | .88 |

A. C-terminal fragments

| | | | | | | |
|---|---|---|---|---|---|---|
| F:30-128 | 0.52 | 2.07 | 0.88* | .88 | >3.5** | >3.5** |
| F:46-128 | 0.34 | .68 | 0.22 | 1.75* | 3.5 | >3.5** |
| F:56-128 | 0.83 | 1.65 | 0.22 | 1.75* | >7** | >7** |
| F:72-128 | 3.5** | 7* | 3.5** | >7** | >3.5** | >3.5** |
| F:84-128 | 1.92** | >3.5** | 1.75 | 3.5* | >3.5** | >3.5** |

B. N-terminal fragments

| | | | | | | |
|---|---|---|---|---|---|---|
| F:1-45 | 0.49 | 0.98 | 0.13 | 0.5 | >7** | >7** |
| F:1-71 | 1.93** | 3.86 | 0.23 | >0.92* | .44 | .88 |
| F:1-83 | 0.43 | 0.87 | 0.34 | 0.69 | .44 | .88 |
| F:1-97 | 0.07 | 0.14 | 0.04 | 0.08 | .44 | .88 |
| F:1-107 | 0.57 | 0.57 | 0.49 | >1.96* | .44 | .44 |

C. Middle fragments

| | | | | | | |
|---|---|---|---|---|---|---|
| F:30-97 | 0.44 | 0.88 | 0.44 | 1.75* | >3.5** | >3.5** |
| F:46-97 | 0.88 | 1.75 | 0.44 | 1.75 | 3.5 | >3.5** |

*p-value <0.05 compared to full-length
**p-value <0.001 compared to full-length

When evaluating the antimicrobial activity of the N-terminal fragments against P. mirabilis, all N-terminal fragments except for F:1-45 had similar or increased activity compared to full-length RNase 7. Unlike its activity against the other two uropathogenic bacteria, F:1:45 demonstrated no activity at high concentrations against P. mirabilis.

The differential activities of both F:1-71 and F:1-97 were confirmed with bacterial viability assays (FIG. 1 and FIG. 2). All N-terminal fragments permeabilized and killed E. coli and S. saprophyticus to a similar extent as full-length protein with the exception of F:1-97 (FIG. 1 and FIG. 2). Comparable to its increased antimicrobial activity, fragment F:1-97 demonstrated increased membrane permeabilization and subsequent death against both strains. Finally, fragment F:1-71 demonstrated similar permeability to full-length against S. saprophyticus but not E. coli, which is consistent with its antimicrobial profile. All permeabilization and antimicrobial assays were confirmed with Live/Dead assays

S. saprophyticus Activity is Mediated by a Different Middle Fragment Domain than E. coli Activity:

The secondary structures of AMPs have been implicated in determining/influencing antimicrobial activity. RNase 7 is known to have 3 α-helices and 2 triple-stranded, antiparallel β-sheets (FIG. 12). Fragments F:30-97 and F:46-97 that were devoid of N-terminal and C-terminal domains were also constructed (FIG. 12). These fragments were constructed to incorporate the first four beta sheets (F: 30-97) and beta sheets 2-4 F: 46-97. The MICs and MBCs of these fragments were two-fold higher than full-length protein. Fragment F:46-97 presents potent membrane penetrating activity against E. coli (FIG. 1 and negligible activity against S. saprophyticus (FIG. 2). Fragment F:30-97 penetrates the membrane of both strains with less activity when compared with full-length RNase 7. These findings were verified with Live/Dead assays.

Full-length RNase 7 Activity Against P. mirabilis Seems to be Exclusively Microbicidal:

The full-length RNase 7 peptide had MIC and MBC values that were identical indicating only concentrations of peptide that killed were effective against P. mirabilis. Because a lower concentration did not inhibit 90% growth (MIC) compared to the 99% killing (MBC), P. mirabilis appears to be resistant to the inhibitory characteristics RNase 7 possesses against the other uropathogens. Furthermore, the N-terminal fragments (other than F:1-45) did demonstrate a MIC at a lower concentration compared to MBC indicating that the C-terminus may play a role in P. mirabilis's ability to be resistant to the inhibitory effects of RNase 7 (Table 1).

Antimicrobial Activities of RNase 7 and Fragments are Diminished at High pH:

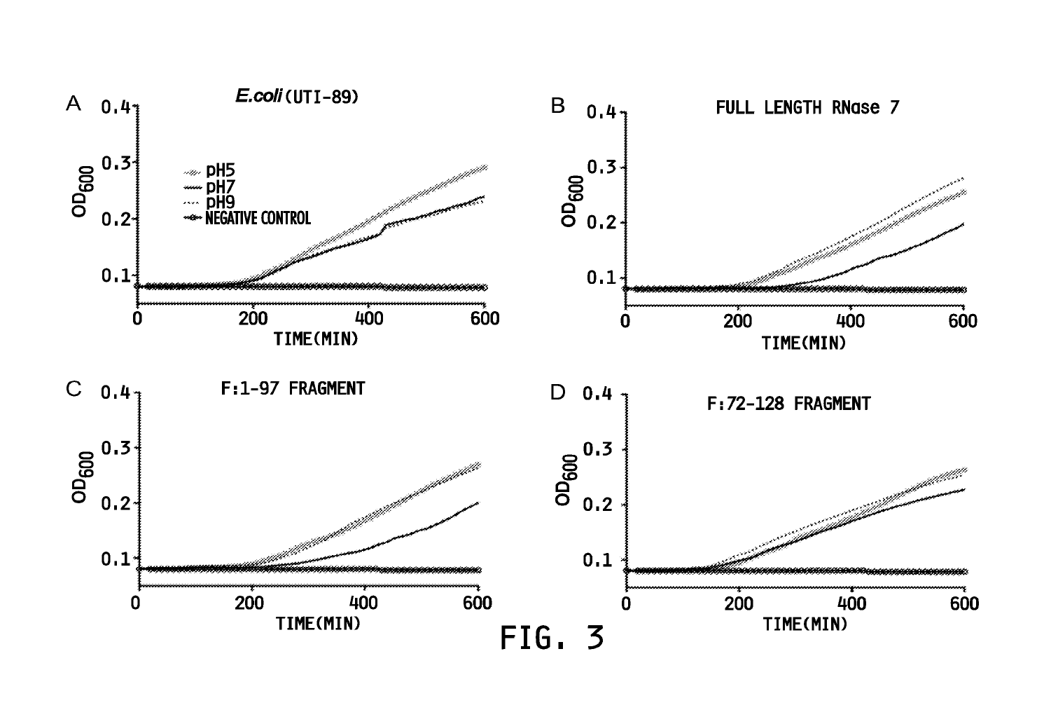

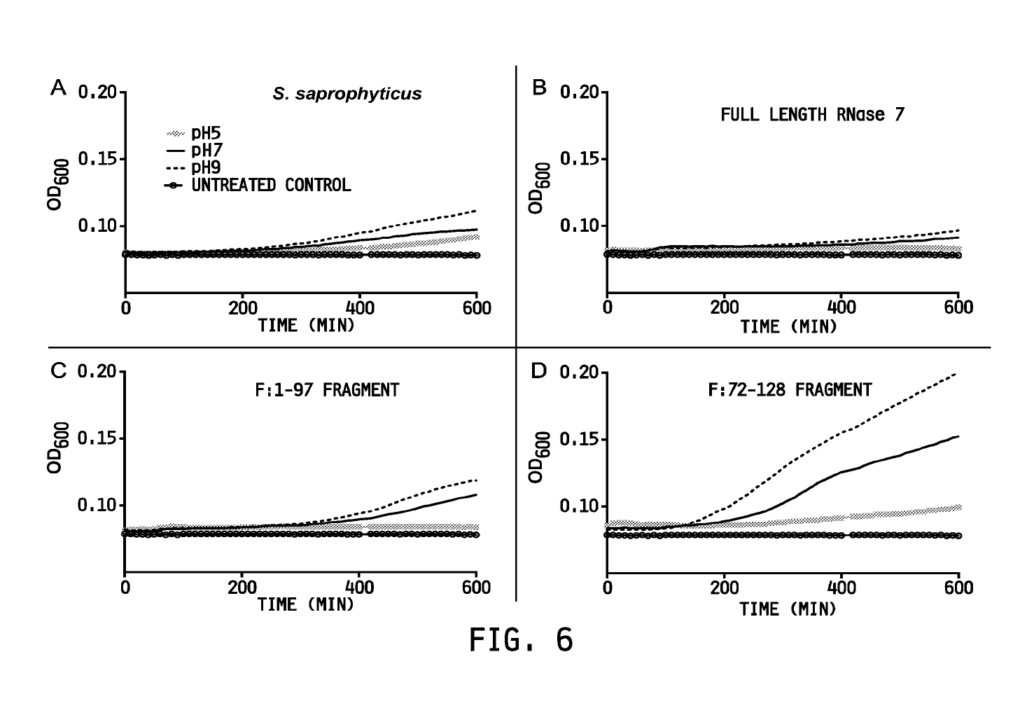

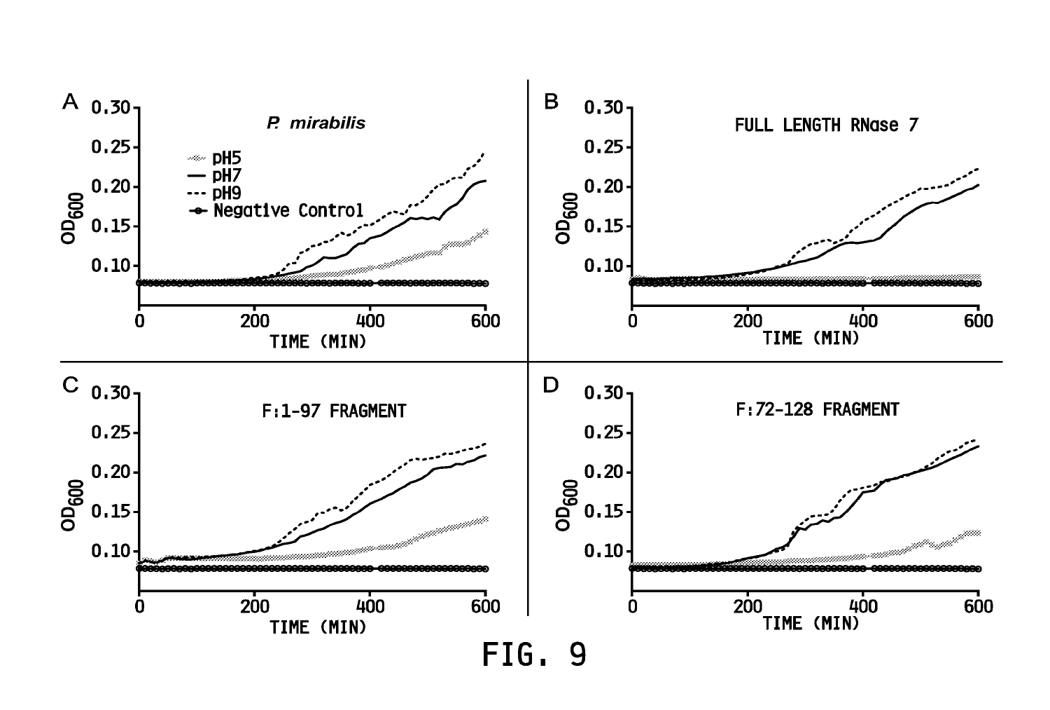

E. coli had the most robust growth in acidic pH of 5, while S. saprophyticus and P. mirabilis grew best in alkaline pH 9 media (FIG. 3A, FIG. 6A, and FIG. 9A). Full-length RNase 7 maintained high activity at pH 5 against S. saprophyticus and P. mirabilis while its activity was optimal at pH 7 against E. coli (FIG. 3B, FIG. 6B, and FIG. 9B). Fragments F:1-97 (high activity fragment) and F:72-128 (low activity fragment) demonstrated a similar pattern with a few exceptions. S. saprophyticus actually grew better with the addition of F:72-128 at pH 7 and 9 (FIG. 6D) compared to control conditions (FIG. 6A).

Sodium Chloride Concentration Affects Activity of RNase 7 Against Certain Uropathogens:

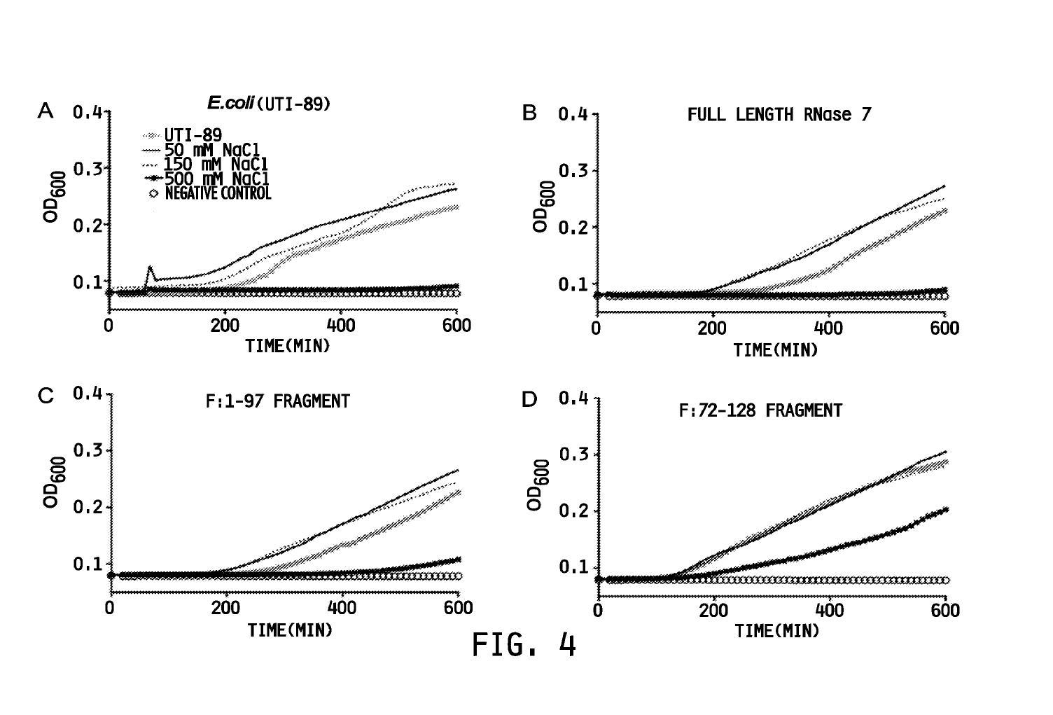

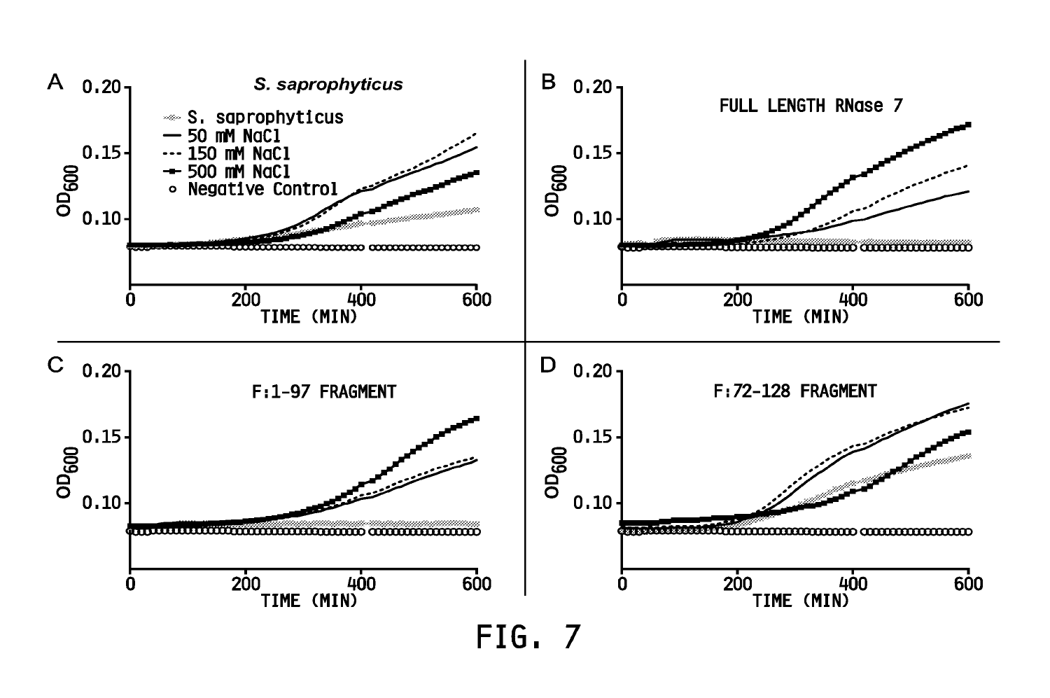

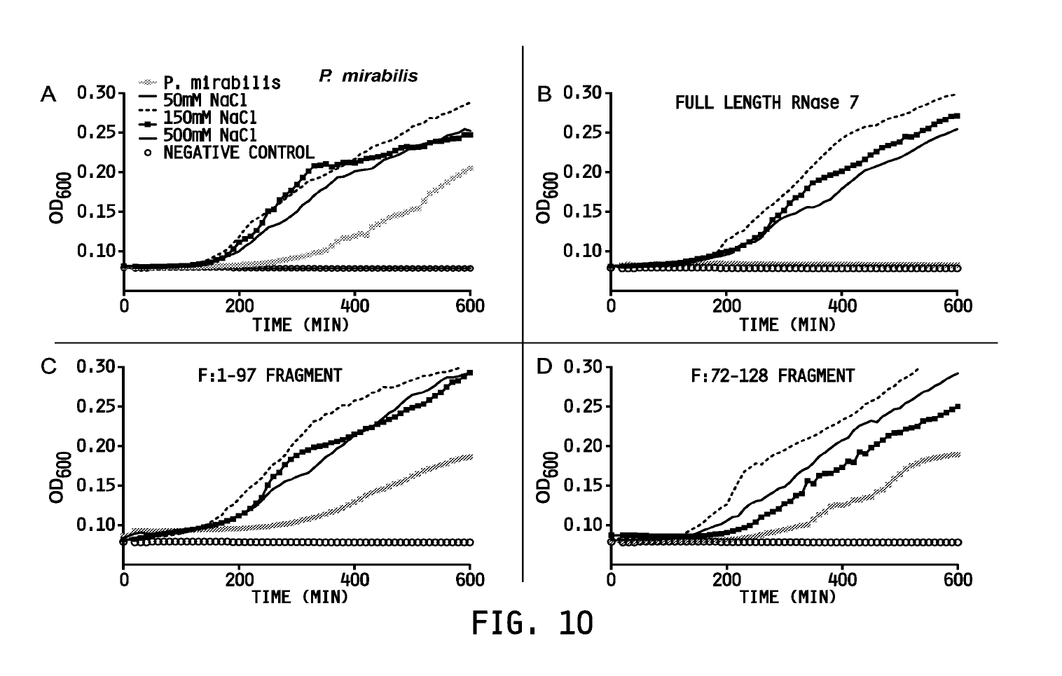

For complete results of the effect of NaCl concentrations, please refer to FIG. 4, FIG. 7, and FIG. 10. NaCl concentration had little effect on peptides against E. coli (FIG. 4). Full-length RNase 7 and fragment F:1-97 activity against S. saprophyticus was diminished in direct proportion to NaCl concentration (FIG. 7B and FIG. 7C). Full-length activity against P. mirabilis was diminished significantly with additional of any NaCl titer (FIG. 10B). Interestingly, the inhibitory effect of 500 mM NaCl against E. coli was diminished with the addition of F:72-128 (FIG. 4D).

Calcium Chloride Concentration Affects Activity of RNase 7 Against all Uropathogens:

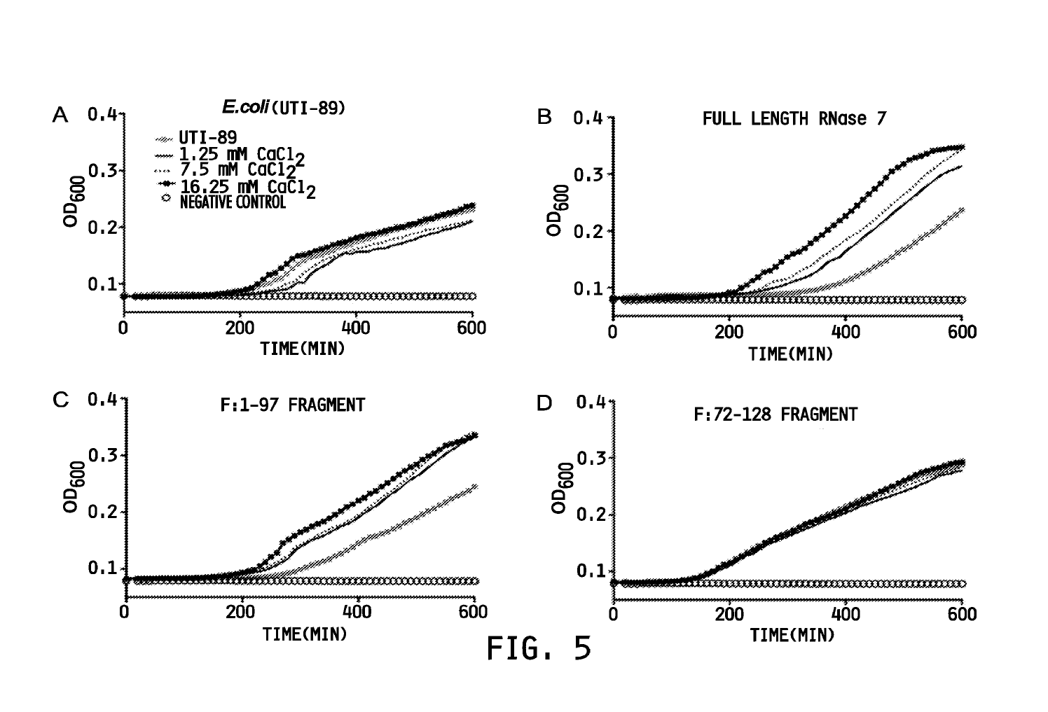

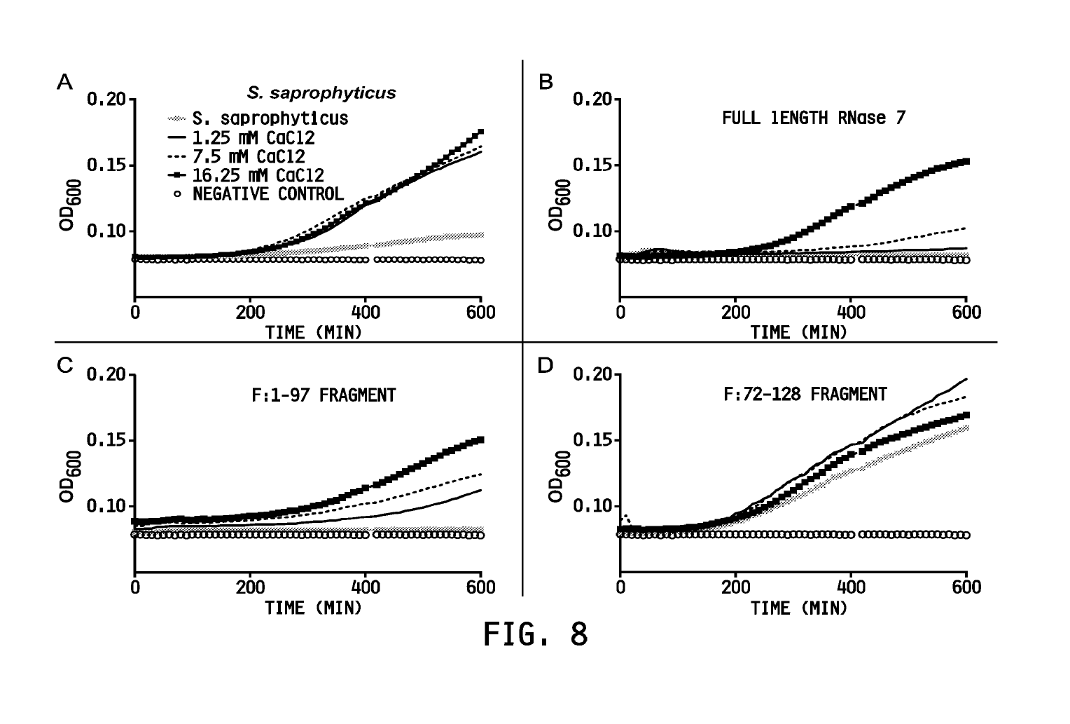

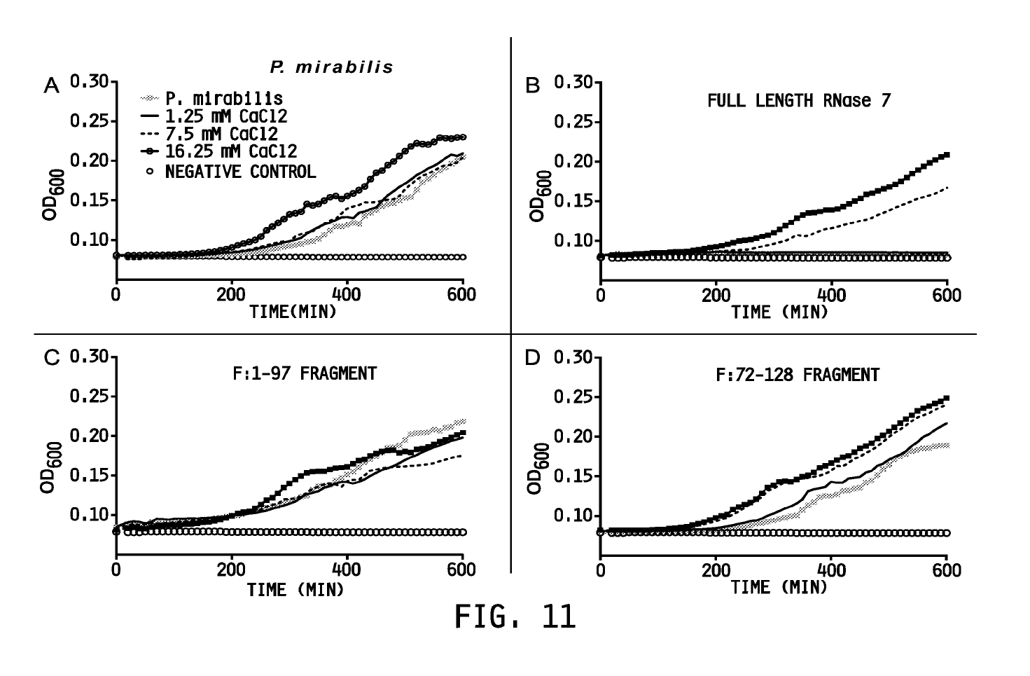

High concentrations of CaCl₂ (7.5 mM and 16.25 mM) significantly reduced full-length RNase 7 activity against all uropathogens (FIG. 5B, FIG. 8B, and FIG. 11B). The potent fragment F:1-97 was also sensitive to CaCl₂ concentrations with the exception of unchanged activity under all conditions against P. mirabilis (FIG. 5C, FIG. 8C, and FIG. 11C). Interestingly, S. saprophyticus grew significantly better with CaCl₂ compared to conditions without CaCl₂ (FIG. 8A).

AMP Fragment Activity is not Entirely Dependent on Net Charge, Hydrophobicity and Hydrophobic Moment:

The fragment primary AMP properties are presented in Table 2. The net charge is comparable for the N-terminal and C-terminal fragments despite the lower activity of the C-terminal fragments, and charge is not associated with antimicrobial activity. Within the N-terminal fragments, F:1-107 had a higher net charge than F:1-97 yet had lower antimicrobial activity. The hydrophobicity was similar between C-terminal and N-terminal fragments despite activity differences. The mean hydrophobic moment was similar between fragments and did not correlate with activity. The maximum hydrophobic moment was higher in the N-terminal fragments than the C-terminal fragments, which correlates with overall activity. On the other hand, within the N-terminal fragments the maximum hydrophobic moment was the same within all fragments despite differential activities. Therefore, this factor may play some role in overall activity, but it does not completely explain antimicrobial activity.

It has been previously reported that RNase 7 is an important AMP in a variety of epithelial tissues including the urinary tract (Spencer, J. D., et al., 2011. Ribonuclease 7 is a potent antimicrobial peptide within the human urinary tract. Kidney Int. 80:174-180). Described herein are the antimicrobial properties of full-length RNase 7 and recombinant fragments against uropathogenic bacteria. Subsequently, our results expand the current knowledge and identify critical regions of the RNase 7 peptide that are crucial to maintaining its antimicrobial properties. Described herein are regions of the RNase 7 molecule that confer differential activity against Gram-positive and Gram-negative uropathogens. Unexpectedly, the antimicrobial properties do not seem significantly linked to peptide charge, amphipathicity or hydrophobicity. Finally, because the urinary environment is so dynamic, the antimicrobial activity of the RNase 7 peptide under physiologic ranges of pH, osmolarity, and ionic concentration was evaluated. The results demonstrate that alterations in these parameters can alter RNase 7's bacteriostatic properties.

The C-terminus of RNase 7 does not appear to contribute significantly to the overall antimicrobial activity. Only the longest C-terminal fragments had activity against S. saprophyticus that were comparable to the full-length peptide. Unexpectedly, F:30-128, F:46-128, and F:56-128 had preservation of inhibitory activity but had diminished killing capability. Furthermore, the fragments that did not include the first 71 amino acids had reduced activity implicating that this region may be important to overall antimicrobial function. To date, the role of the C-terminal fragment is not completely clear. Without being bound by theory, it is believed, that this part of the molecule may be important in peptide stability in eukaryotic cells and possibly even important in trafficking to the cell surface or secretory vesicles during infection. This preservation of inhibitory activity (low MIC) with loss of killing activity (relatively high MBC) against E. coli and S. saprophyticus coupled with the fact that only fragments lacking the C-terminus had inhibitory activity against P. mirabilis (MIC=MBC for all other peptides) suggests that the C-terminus may be critical for inhibiting microbial growth and division but not critical to actual killing.

The discovery of the importance of the N-terminal portion of RNase 7 in antimicrobial functions is described herein. Interestingly, fragment F:1-71 has markedly reduced activity against E. coli but not S. saprophyticus and P. mirabilis bacteria compared to full-length peptide. By adding an additional 12 amino acids to this fragment (F:1-83) partially restores the antimicrobial activity. These additional 12 amino acids add another secondary motif. The smaller N-terminal fragment (F:1-45) has similar E. coli activity to F:1-83. Further studies are necessary to elucidate the mechanisms of fragment F:1-71's decreased antimicrobial activity as this information will offer significant insight into structure-function relationships of AMPs.

Secondary structure appears to play a critical role in determining the functional properties of RNase 7. Our data suggests that conservation of beta-sheets 1, 3 and 4 is critical to overall antimicrobial activity. Disrupting the peptide domain located at amino acids 71 and 72 leads markedly reduced antimicrobial activity presumably from disrupting the interaction between beta-sheet 1 and 3. Fragments F:1-71 and F:72-128 have MICs and MBCs that significantly differ from the fragments that include the amino acids 71 and 72. Furthermore, F:84-128, which does not contain the N-terminal elements or these critical amino acids, has poor antimicrobial function against all uropathogens.

Differential activity of recombinant peptides under different conditions against uropathogenic bacteria is described herein. It is herein described that altered pH, NaCl, and CaCl₂ concentrations are important to maximizing antimicrobial activity of the peptides and compositions described herein. For example, patients with hypercalciuria are at increased risk for UTIs. Therapies that combine treatment with the peptides and/or compositions described herein with regimes that lower calcium levels in the urine are contemplated.

Consequently, hypercalciuric patients with “ideal” urinary conditions may be less susceptible to UTIs by having optimal antimicrobial peptide activity. Further, optimizing these patients urinary conditions during infections could help with clearance of uropathogens if infections do occur. Clearly, further investigations are needed to elucidate these relationships and could serve as the foundation for clinical studies in the future.

Another important concept in AMP antimicrobial activity involves peptide properties such as net charge, isoelectric point and hydrophobicity. Net charge has been implicated in cationic AMPs attraction to the negatively charged bacterial membrane. Classically, AMPs with a higher net positive charge have been associated with increased antimicrobial activity. Our recombinant peptides did not demonstrate any strong correlation between net charge and antimicrobial activity or membrane permeability (Table 1 and 2). Furthermore, N-terminal fragments and full-length RNase 7 had the highest amphipathicity values, but their values did not differ from one another. These findings suggest that peptide properties may contribute to antimicrobial activity but do not dictate specific activities.

In summary, RNase 7 is an extremely potent antimicrobial peptide that plays a significant role in maintaining the sterility of the kidney and urinary tract. It has been have previously described that uropathogenic bacteria are susceptible to RNase 7 at very low concentrations. The susceptibility and broad-spectrum activity presented in this study makes this molecule an interesting candidate to develop as a therapeutic to treat urinary tract infections.

TABLE 2
Fragment AMP properties

| | Net | | Max | Mean |
|---|---|---|---|---|
| Fragment | Charge | Hydrophobicity | μH* | μH* |
| RNase 7 | 16 | −0.93 | 0.727 | 0.266 |

A. C-terminal fragments

| | | | | |
|---|---|---|---|---|
| F:30-128 | 12 | −0.9 | 0.602 | 0.283 |
| F:46-128 | 9 | −0.88 | 0.468 | 0.229 |
| F:56-128 | 10 | −1.03 | 0.468 | 0.234 |
| F:72-128 | 8 | −0.94 | 0.415 | 0.194 |
| F:84-128 | 7 | −1.23 | 0.415 | 0.23 |

B. N-terminal fragments

| | | | | |
|---|---|---|---|---|
| F:1-45 | 7 | −1.16 | 0.727 | 0.271 |
| F:1-71 | 8 | −1.03 | 0.727 | 0.318 |
| F:1-83 | 9 | −0.84 | 0.727 | 0.299 |
| F:1-97 | 13 | −1.02 | 0.727 | 0.28 |
| F:1-107 | 15 | −0.97 | 0.727 | 0.273 |

C. Middle fragments

| | | | | |
|---|---|---|---|---|
| F:30-97 | 9 | −1 | 0.602 | 0.275 |
| F:46-97 | 6 | −0.99 | 0.498 | 0.233 |

*μH = hydrophobic moment as measure of amphipathicity

The complete disclosure of all patents, patent applications, and publications, and electronically available materials cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. An antimicrobial peptide comprising a contiguous fragment of SEQ ID NO: 1, wherein said fragment consists of
i) an amino acid sequence selected from amino acids 1-107 of SEQ ID NO: 1;
ii) a peptide having at least 90% amino acid sequence identity with SEQ ID NO: 5 or SEQ ID NO: 7; or
iii) a peptidomimetic derivative of i) or ii), with the proviso that the antimicrobial peptide does not comprise a 10 amino acid sequence that is identical to a contiguous 10 amino acid sequence present in amino acids 108-128 of SEQ ID NO: 1.

2. The antimicrobial peptide of claim 1 wherein said peptide has at least 90% or 95% sequence identity to an amino acid sequence of SEQ ID NO: 2, SEQ ID NO: 4, SEQ ID NO: 5, SEQ ID NO: 6 or SEQ ID NO: 7, or a peptidomimetic derivative of SEQ ID NO: 2, SEQ ID NO: 4, SEQ ID NO: 5, SEQ ID NO: 6 or SEQ ID NO: 7.

3. The antimicrobial peptide of claim 1 wherein said fragment consists of SEQ ID NO: 5 or SEQ ID NO: 7, or a peptide that differs from SEQ ID NO: 5 or SEQ ID NO: 7 by 1 to 8 amino acid substitutions.

4. The antimicrobial peptide of claim 3 wherein the amino acid substitutions are conservative amino acid substitutions.

5. A composition comprising the antimicrobial peptide of claim 1 and a pharmaceutically acceptable carrier.

6. The composition of claim 5 further comprising a supplemental anti-microbial agent.

7. A method for treating a subjecting having a bacterial infection by administering a therapeutically effective amount of the composition of claim 5 to the subject.

8. The method of claim 7, wherein the subject is human.

9. The method of claim 7, wherein said composition comprises two or more RNase 7 peptide fragments selected from the group consisting of SEQ ID NO: 2, SEQ ID NO: 4, SEQ ID NO: 5 and SEQ ID NO: 6.

10. The method of claim 7, wherein the bacterial infection is caused by a gram-positive bacteria.

11. The method of claim 7, wherein the bacterial infection is caused by a gram-negative bacteria.

12. The antimicrobial peptide of claim 1 wherein the peptide fragment of SEQ ID NO: 1 includes at least 45 amino acids.

13. The antimicrobial peptide of claim 1 wherein the peptide fragment of SEQ ID NO: 1 includes at least 95 amino acids, but no more than 107 amino acids.

14. A method for treating or preventing a bacterial infection in a patient, said method comprising the step of, administering to the patient an effective amount of a composition comprising the antimicrobial peptide of any one of claim 1 to 4, 12 or 13.

15. The method of claim 14 wherein the infection is a urinary tract infection.

16. The method of claim 14 wherein the composition is administered on or in a biomedical device.

17. The method of claim 16 wherein the medical device is selected from the group consisting of staples, clips, drug delivery devices, stents, catheters, sutures, woven mesh, gauze, dressings, and growth matrices.

18. The method of claim 17 wherein the biomedical device is a selected from the group consisting of a catheter and a stent.

19. The method of claim 15 wherein the composition is administered directly into the bladder.

20. The method of claim 15 wherein the infection is caused by an organism selected from the group consisting of Escherichia, Staphylococcus, Proteus Klebsiella, Enterococcus, Proteus, Morganella, Pseudomonas, Group B Streptococcus, and Candida.

21. The method of claim 20 wherein the organism is selected from the group consisting of E. coli, P. aeruginosa, K. pneumonia, P. mirabilis, E. faecalis, and S. saprophyticus.

22. The method of claim 20 wherein the organism is selected from the group consisting of from E. coli, P. mirabilis, and S. saprophyticus.
